SEQUENCE LISTING (33 sequences)

SEQ ID NO: 1 - DNA, 2554 bp, Arabidopsis thaliana
  Promoter (1)..(2554): Fragment of promoter and 5′-untranslated region of the Arabidopsis thaliana gene locus At5g33370
gtcgacaggaaattttagtttcttttaagtgtgcaagaaactgaggataaatttaggtttaaactattagaaaaacctatttgaccaatatattaaaataagaaatagatcaactaactttgttgtagatatactttttcgagttttctttgttttttaatgaatatgttaccaatctcatattgaagagtgacttaatggatattgtggagaaaagaaatcagagattttatgcgggagtagttagttgagatcacacaaaataatcgaaattttaaaaacaaagaatcaagatgcaaaatatctttcattaaatatgataaatcttaagaatttaattcaatagcattcttcggtaaatattgtagtaaactcattatttattttccaaatgtgttcaaaagaaaaatatagattcttatatgttcaaatatttataatatttgaaattttgtaaagttttatattataaatatttaaaacgttttataatattttaattttgaaaaaagtttgattattaattataggatatttaaatgtaaaaatctccaggaatggagattgatgattatgggttttagctataaagttgttggttaaaatggactatgcagagaaaatcattagagcaaatttcagaatatgaaataaatgaaaataagaaaagctggaatttagattgtgattatgaatttaagtaagatttgcatctactgaggcacaaatattcgttaaattgcgtatgcttatcttgcgtatgcttatcttgcgtgaggatcaatataaatatggttagttgtgtttaaatataatccttgagattttgtaatatattaaaattaataggaccgataaaggaaataattattaaaagtatttttaatgtaaaagtcgaattgtttaaatttgttaagacaatatctgcttgagataaattttgtaaggactaagaagaaataaatatttatttgctggattttacagtcaatatatatcgtgttagtcttgttaattaattttgatataaaccaaaactaaaattgtcttgtttagccaactaaaattgtcagttaattaatattctgcagccgatcagatcattaacttatattactaaactaatgttacagctacatttattgtacctacaattcaaattttaaagccctaaattttatagtaattatgtgaacaaatatgcaacaaaaattctacagaaataaatttatacatgtgaacacccatttttctctctagatggttgcaaaaccttccaaatatcaaaggaaaaaagaaaaacaaaaagcaacctgaatgtcatagtcattgtcagaatctaactctgcggctctgggagtaaatctgaacaaagtacttcaaataattctatcctattcccaaagggacctgtatttcctttttacaaccaaattttttatccaattaaaacgccaaaattgaagcgcatgaaaatgttggcatgaaattaaacttctccgtgtctctgggtctaccacatcatgtcttttaatctcgcccccaaaagtagttttaactttttccttaactactctaaaaatgtatatgtgtaaattatgacttttactgagttcttttttttcatctaatagttttgattaataattatatagatatagttctatattctagttaattattttgtatgcccctagttgaattagtagttgaattagtagttgaactagtatactatgataatgataatttcgtttctgcaatattaaaaatgataatttcgttttaaatatttatgcacttaatacattctcagatatgcataattattagcaatatatatgatgttgacagggaattccaaatagattccattttgaaagttacaaaaatgttgaatgtttcttatggtttctctgagtttgttactctcactgtttagatgagtcacttcaataattcgatgcacaagctcacatttatttttgataaaaaatatataatatggtatttatattcttacacaaattcgagagtggattaggcaaacgctgataaatgaaaaattctagtaaatggatgaatagttaggttgataaattgtatagttgttattatatatagcaaattaaagaaaagtactgtaatatggatgaaaaatataacactttatttaataaataataataaaaaaactttaccatcatcacttatttattctcaaccaaaatcacaactctagtacatttagagttgtctccctttaaaatcaatctaacaccaataggtaaaagctggtgaagacctccattaatgcccccattttcctctctattactttttacatatgcacctatatatatagagacaatcatgttctcttctacattctcatttcatagcaaaacaccaaaacagagttcacagaaacatattcaaagatttttcacaaatattaccattttaaatctataaacacccgggtaatgataatttcgtttctgcaatattaaaaatgataatttcgttttaaatatttatgcacttaatacat

SEQ ID NO: 2 - DNA, 1541 bp, Arabidopsis thaliana
  Promoter (1)..(1541): Functionally equivalent fragment of promoter and 5′-untranslated region of the Arabidopsis thaliana gene locus At5g33370
attttgtaaggactaagaagaaataaatatttatttgctggattttacagtcaatatatatcgtgttagtcttgttaattaattttgatataaaccaaaactaaaattgtcttgtttagccaactaaaattgtcagttaattaatattctgcagccgatcagatcattaacttatattactaaactaatgttacagctacatttattgtacctacaattcaaattttaaagccctaaattttatagtaattatgtgaacaaatatgcaacaaaaattctacagaaataaatttatacatgtgaacacccatttttctctctagatggttgcaaaaccttccaaatatcaaaggaaaaaagaaaaacaaaaagcaacctgaatgtcatagtcattgtcagaatctaactctgcggctctgggagtaaatctgaacaaagtacttcaaataattctatcctattcccaaagggacctgtatttcctttttacaaccaaattttttatccaattaaaacgccaaaattgaagcgcatgaaaatgttggcatgaaattaaacttctccgtgtctctgggtctaccacatcatgtcttttaatctcgcccccaaaagtagttttaactttttccttaactactctaaaaatgtatatgtgtaaattatgacttttactgagttcttttttttcatctaatagttttgattaataattatatagatatagttctatattctagttaattattttgtatgcccctagttgaattagtagttgaattagtagttgaactagtatactatgataatgataatttcgtttctgcaatattaaaaatgataatttcgttttaaatatttatgcacttaatacattctcagatatgcataattattagcaatatatatgatgttgacagggaattccaaatagattccattttgaaagttacaaaaatgttgaatgtttcttatggtttctctgagtttgttactctcactgtttagatgagtcacttcaataattcgatgcacaagctcacatttatttttgataaaaaatatataatatggtatttatattcttacacaaattcgagagtggattaggcaaacgctgataaatgaaaaattctagtaaatggatgaatagttaggttgataaattgtatagttgttattatatatagcaaattaaagaaaagtactgtaatatggatgaaaaatataacactttatttaataaataataataaaaaaactttaccatcatcacttatttattctcaaccaaaatcacaactctagtacatttagagttgtctccctttaaaatcaatctaacaccaataggtaaaagctggtgaagacctccattaatgcccccattttcctctctattactttttacatatgcacctatatatatagagacaatcatgttctcttctacattctcatttcatagcaaaacaccaaaacagagttcacagaaacatattcaaagatttttcacaaatattaccattttaaatctataaaca

SEQ ID NO: 3 - DNA, 668 bp, Arabidopsis thaliana
  Promoter (1)..(668): Functionally equivalent fragment (668 bp) of promoter and 5′-untranslated region of the Arabidopsis thaliana gene locus At5g33370
tctcagatatgcataattattagcaatatatatgatgttgacagggaattccaaatagattccattttgaaagttacaaaaatgttgaatgtttcttatggtttctctgagtttgttactctcactgtttagatgagtcacttcaataattcgatgcacaagctcacatttatttttgataaaaaatatataatatggtatttatattcttacacaaattcgagagtggattaggcaaacgctgataaatgaaaaattctagtaaatggatgaatagttaggttgataaattgtatagttgttattatatatagcaaattaaagaaaagtactgtaatatggatgaaaaatataacactttatttaataaataataataaaaaaactttaccatcatcacttatttattctcaaccaaaatcacaactctagtacatttagagttgtctccctttaaaatcaatctaacaccaataggtaaaagctggtgaagacctccattaatgcccccattttcctctctattactttttacatatgcacctatatatatagagacaatcatgttctcttctacattctcatttcatagcaaaacaccaaaacagagttcacagaaacatattcaaagatttttcacaaatattaccattttaaatctataaaca

SEQ ID NO: 4 - DNA, 2103 bp, Arabidopsis thaliana
  Promoter (1)..(2103): Fragment of promoter and 5′-untranslated region of the Arabidopsis thaliana gene locus At5g22430
ccggttgatatggtcgtaaacgcaatgattgcaatcgcagctgaccattgccatgattctggaagtcatacagtttaccatgtcggctcatctaaccaaaatccggtgatatataaacaaatttatgagatgatgagtcgttatttcatgaaaagccctcttgtcggacgtaatggtatgcttatagttccaaaagttacaagaatatccacactggctaggttccgtgtatacacaaatcttcgctataaattacctatacaggtaacagcttgcataaattgcacacgatttttttttttttctgtttgagtgttattctttttattattttaatagcaacctctcattttggctttgtgttttttcttctttagatattgggattacttagtgttatttccctatcccaaagagataaatttgcacttcacaaccgtaaattcaagatggccatgagattggtgaaactttacaagccttatgtactcttcaagggcatgtatgtatcactctcatatttcctccaacattccttttttacaagtattttcttccacttttaatgtatctagtagatatttttcatgccttagttcacgttttaactatagtttgaaattctattgtgtattcaatgcagatttgatgataaaaacatggaaactttacgtatcaagaatgaagccaaagatatggagaaattgtttggcaccaaccccaaatgcattgattgggaagactattttatgatacgcatatccctggcctcatgacacacgtattcaagaaatgaacttcaaatacaagttaatatacgaagtcaagtccaaagatattgttttgcatgggatgaagcctactatatataattactaagtatataaataataaatcgatggaaaatcgacgacaagtaatgttgtctttgtttgcttcttaatgtataatccatatttggaatttgcctatctaaaatccctcggcttgtctttggttatatataaaattggactactcattttgacaagtaaacatatactactttttaagtcaagaacatatactacttttccgacagaatcccacatttctttagttcttttactttgataagcaacgtcatcacttttaatgatattttaagtgaatgaaaaagagatgttgagtgaaggaaaaaaaagaagaaaaacaactagacgtaagtaaaattggactactcattttgacaagtaaaggtgggcttcggtgaagcaagaggttacaaagcaaatggacatcaaccttaaacactagtatataaaaagattttacaagtgacctgcgcttgtagttacctagttcagctaaaagtaaaaaaatgacacaaatgtaatggacaagtactgaaagataattttattggatataaatgatgtaaccactgacactgttgagcaaaccttcacggctcattaaagatttataaaggacagtaagaaaaaaaatatgtttccccactaggctttctatttttagtatcactctaaactctagcgagtgaagatggctaaagtatagctagttaatgcttaatgcgtaatataaatatagttggtatatgattaaatactagtaagtctggttttcaagtaatgcagtttaaatggttcactgatgaattaaaattgacacacaataaattgtttaaacataatataaatgaggaaattaatgttttgaaggaacttcaatacagtataagaatatatgtgtttctcaaaatggtgatttatgtatagttttctagggtctctccttaactccgttgctagacataattggtgttcgtgtataaatgatttgaatctaacaaattgttaggttgaataaaattttgcagcaaatttgaaggtggatgaatttaagagccgttaatatagaagaactgcaaaagtacgttatctatttgtgtaaagccaaccaccaaaaaacaacgcattttggttcctttaataatcatttctttcttttatataaacctaagatacgttccccatatttcactcactcaagaagacatctaaagccatcaaaaatatatccatagaagcttcatcgaaa

SEQ ID NO: 5 - DNA, 1376 bp, Arabidopsis thaliana
  Promoter (1)..(1376): Functionally equivalent fragment of promoter and 5′-untranslated region of the Arabidopsis thaliana gene locus At5g22430
cattgattgggaagactattttatgatacgcatatccctggcctcatgacacacgtattcaagaaatgaacttcaaatacaagttaatatacgaagtcaagtccaaagatattgttttgcatgggatgaagcctactatatataattactaagtatataaataataaatcgatggaaaatcgacgacaagtaatgttgtctttgtttgcttcttaatgtataatccatatttggaatttgcctatctaaaatccctcggcttgtctttggttatatataaaattggactactcattttgacaagtaaacatatactactttttaagtcaagaacatatactacttttccgacagaatcccacatttctttagttcttttactttgataagcaacgtcatcacttttaatgatattttaagtgaatgaaaaagagatgttgagtgaaggaaaaaaaagaagaaaaacaactagacgtaagtaaaattggactactcattttgacaagtaaaggtgggcttcggtgaagcaagaggttacaaagcaaatggacatcaaccttaaacactagtatataaaaagattttacaagtgacctgcgcttgtagttacctagttcagctaaaagtaaaaaaatgacacaaatgtaatggacaagtactgaaagataattttattggatataaatgatgtaaccactgacactgttgagcaaaccttcacggctcattaaagatttataaaggacagtaagaaaaaaaatatgtttccccactaggctttctatttttagtatcactctaaactctagcgagtgaagatggctaaagtatagctagttaatgcttaatgcgtaatataaatatagttggtatatgattaaatactagtaagtctggttttcaagtaatgcagtttaaatggttcactgatgaattaaaattgacacacaataaattgtttaaacataatataaatgaggaaattaatgttttgaaggaacttcaatacagtataagaatatatgtgtttctcaaaatggtgatttatgtatagttttctagggtctctccttaactccgttgctagacataattggtgttcgtgtataaatgatttgaatctaacaaattgttaggttgaataaaattttgcagcaaatttgaaggtggatgaatttaagagccgttaatatagaagaactgcaaaagtacgttatctatttgtgtaaagccaaccaccaaaaaacaacgcattttggttcctttaataatcatttctttcttttatataaacctaagatacgttccccatatttcactcactcaagaagacatctaaagccatcaaaaatatatccatagaagcttcatcgaaa

SEQ ID NO: 6 - DNA, 746 bp, Arabidopsis thaliana
  Promoter (1)..(746): Functionally equivalent fragment of promoter and 5′-untranslated region of the Arabidopsis thaliana gene locus At5g22430
aaaatgacacaaatgtaatggacaagtactgaaagataattttattggatataaatgatgtaaccactgacactgttgagcaaaccttcacggctcattaaagatttataaaggacagtaagaaaaaaaatatgtttccccactaggctttctatttttagtatcactctaaactctagcgagtgaagatggctaaagtatagctagttaatgcttaatgcgtaatataaatatagttggtatatgattaaatactagtaagtctggttttcaagtaatgcagtttaaatggttcactgatgaattaaaattgacacacaataaattgtttaaacataatataaatgaggaaattaatgttttgaaggaacttcaatacagtataagaatatatgtgtttctcaaaatggtgatttatgtatagttttctagggtctctccttaactccgttgctagacataattggtgttcgtgtataaatgatttgaatctaacaaattgttaggttgaataaaattttgcagcaaatttgaaggtggatgaatttaagagccgttaatatagaagaactgcaaaagtacgttatctatttgtgtaaagccaaccaccaaaaaacaacgcattttggttcctttaataatcatttctttcttttatataaacctaagatacgttccccatatttcactcactcaagaagacatctaaagccatcaaaaatatatccatagaagcttcatcgaaa

SEQ ID NO: 7 - DNA, 2945 bp, Arabidopsis thaliana
  Promoter (1)..(2945): Fragment of promoter and 5′-untranslated region of the Arabidopsis thaliana gene locus At1g26630
gattttatatataacagagctagtacaacgagtacaaaagtggaaacaaccacttaacaaaaggcttacctaataactattacagcagatgattatcagaagtatacataacaaaccattgtaacaggaggaagtaaaacttgtatcataaagtgagttctaagcagacaatcataaacttgtatcataaagtgagttctaagcagacaatcataaacttgtatcataatattggtaagtatttagaacaaataaaatgttgagttttatacgaaaacatggtattttatctgatgtcatgttgctttctataaacttccattcatacatggctactattttacacgacaccactctagtttatccgactatacattatttaataaactagtaagtaattaacaatcatgtgcgatggaaaatgagatttaggtacccacatatttatgattttttttattgagatttattttgcaaatcagttcaaatttttggtttttagcatgttagttcaataattaacaaagaaattaaacatttgtatgtacaataacattttatgaggaaattaacatatgacaaagttctagtcgataataagttcatgcctttcaaacaagatgaaaaaggatatttagcaaaaaattgatatgtctaagtttattcaagtttttagttttctaaggacaattaatcatgctcaattgctcataatgaaatgcatcatatcattatatgatttggttttgcttataaatcataaatcataaatgacgtttcccttacgaaaaatatcaaatttttatatatgaaagataatgggccgtaaatgatcagtcccatatagcccattccaagtacgcaactcatttctggagatgttcgtggaggttttcgaatttggttttgtttcccaaatacccaaaatatcatcaagcataatgggccgtaaatgctctttgatttagcttcctaaaagcccaaaatagcatcaagtcttagtctatagggctataattgtcaaaatgagccagctcgctcagctcagctcacctcatgatgaacttggatcttttatgagccagctcagctcaactcatttattatatgagcttcaaaatacaaactcggactcagatcatctaaatcacgagctaaatgagctagctcgcgagctaatgcgaataaatgaaaaaatttatattttctaaacttgttatgtaaattatatatttttaaaaatatatttctatttttatagattatataaatatttatgttttgattattgactttttgtgtatattactaatatttattttcagaaattgttatgtaacttctatattttctaaaatatatttctattatttatagaaaaatatatatatactttgattgttagctttttgtatatattacttcaaaaaaaccaagctcatgttcattaaagctcgctcatataactcgtgagctaaataaagctcgatcactaaactcatttattaaatgatccttaaaaatagaactcgcgctcatgaataactgagccgagttgagccagctcatgagctatcagcttattttgacacccctagctataaccaaaactgaagatctcttgagtgttgcatcaaactactatttttaatcaatagttagcttttttttgttatattatcaattaaatattaacttttatttgattaaatatttatttaaatttaattttgattttaaaatttatcgtgcaaaatttagactaaaaagttatatactcaattgtttttcatattcgtttcttatgaaattcatatactcatgtgtattggatttaattgtagtgatttattttgtaatgaaaaagttaatatttttaattaataatacaaaaaaataatttctttactatatcttaaaaattttaaattattttttttacattttaagtttagttattaaatcaaaaaataattctagatttcatgtaactgaatttttttttttgaaaattaaatctcgcctttagagtaaaaaaaaactctatttttttagttatgtagtctgtaatatgaggaattagatgtcacaagctaaagtggggccgcatggaatgcacgcgagagatgcaagagagagaaaaaaataaaatcgggaaaatctaaatcgttgagagattataatccaacagctgagaaataggataaattatcaaactagagaaactttggcagctttggatgctgagaatgagtaaatgtcaatgaatgagaaggcaaacaattaaggagaagcaacacaaagtgaaacacaatctactcaagcagctactacggtatttaactctaataaacgcttaaagtttgatgttcggaaagagtttagacccatgttagaagttgtttagattttttcactacttttgtatttctgttttaaattagaatctttgatgaaattctgttttgtataatcttttaaattattaatgtttgagtgaattttggatttataaatttgttagaaactttgattaaattaaagaattaaaatcaattaagggtaacaaataaagttttttatacaaaatacaaaatacatgattcaatttaaatacatatgacagtgaatataatatatatgtcaaatacagaaataaatacaatttaaaatatctatataaaatacagtttttttgaatccttttttttttatgaagaggcctgttttgagtttgggctgggctgaattgtttttacccaccacaacattcctaaggataataatacagtaaccctagaaaggtttcctccaccttcctcttcccctcctatataaaaaaaatcgacatcgcttttgctcacttctctctcttaggttttttttcccttctcccaatctcatcttctccgaaaacctttcttctctcaaatttctgtgaaaac

SEQ ID NO: 8 - DNA, 1628 bp, Arabidopsis thaliana
  Promoter (1)..(1628): Functionally equivalent fragment of promoter and 5′-untranslated region of the Arabidopsis thaliana gene locus At1g26630
attttcagaaattgttatgtaacttctatattttctaaaatatatttctattatttatagaaaaatatatatatactttgattgttagctttttgtatatattacttcaaaaaaaccaagctcatgttcattaaagctcgctcatataactcgtgagctaaataaagctcgatcactaaactcatttattaaatgatccttaaaaatagaactcgcgctcatgaataactgagccgagttgagccagctcatgagctatcagcttattttgacacccctagctataaccaaaactgaagatctcttgagtgttgcatcaaactactatttttaatcaatagttagcttttttttgttatattatcaattaaatattaacttttatttgattaaatatttatttaaatttaattttgattttaaaatttatcgtgcaaaatttagactaaaaagttatatactcaattgtttttcatattcgtttcttatgaaattcatatactcatgtgtattggatttaattgtagtgatttattttgtaatgaaaaagttaatatttttaattaataatacaaaaaaataatttctttactatatcttaaaaattttaaattattttttttacattttaagtttagttattaaatcaaaaaataattctagatttcatgtaactgaatttttttttttgaaaattaaatctcgcctttagagtaaaaaaaaactctatttttttagttatgtagtctgtaatatgaggaattagatgtcacaagctaaagtggggccgcatggaatgcacgcgagagatgcaagagagagaaaaaaataaaatcgggaaaatctaaatcgttgagagattataatccaacagctgagaaataggataaattatcaaactagagaaactttggcagctttggatgctgagaatgagtaaatgtcaatgaatgagaaggcaaacaattaaggagaagcaacacaaagtgaaacacaatctactcaagcagctactacggtatttaactctaataaacgcttaaagtttgatgttcggaaagagtttagacccatgttagaagttgtttagattttttcactacttttgtatttctgttttaaattagaatctttgatgaaattctgttttgtataatcttttaaattattaatgtttgagtgaattttggatttataaatttgttagaaactttgattaaattaaagaattaaaatcaattaagggtaacaaataaagttttttatacaaaatacaaaatacatgattcaatttaaatacatatgacagtgaatataatatatatgtcaaatacagaaataaatacaatttaaaatatctatataaaatacagtttttttgaatccttttttttttatgaagaggcctgttttgagtttgggctgggctgaattgtttttacccaccacaacattcctaaggataataatacagtaaccctagaaaggtttcctccaccttcctcttcccctcctatataaaaaaaatcgacatcgcttttgctcacttctctctcttaggttttttttcccttctcccaatctcatcttctccgaaaacctttcttctctcaaatttctgtgaaaac

SEQ ID NO: 9 - DNA, 587 bp, Arabidopsis thaliana
  Promoter (1)..(587): Functionally equivalent fragment of promoter and 5′-untranslated region of the Arabidopsis thaliana gene locus At1g26630
aaagtttgatgttcggaaagagtttagacccatgttagaagttgtttagattttttcactacttttgtatttctgttttaaattagaatctttgatgaaattctgttttgtataatcttttaaattattaatgtttgagtgaattttggatttataaatttgttagaaactttgattaaattaaagaattaaaatcaattaagggtaacaaataaagttttttatacaaaatacaaaatacatgattcaatttaaatacatatgacagtgaatataatatatatgtcaaatacagaaataaatacaatttaaaatatctatataaaatacagtttttttgaatccttttttttttatgaagaggcctgttttgagtttgggctgggctgaattgtttttacccaccacaacattcctaaggataataatacagtaaccctagaaaggtttcctccaccttcctcttcccctcctatataaaaaaaatcgacatcgcttttgctcacttctctctcttaggttttttttcccttctcccaatctcatcttctccgaaaacctttcttctctcaaatttctgtgaaaac

SEQ ID NO: 10 - DNA, 2572 bp, Arabidopsis thaliana
  Promoter (1)..(2572): Fragment of promoter and 5′-untranslated region of the Arabidopsis thaliana gene locus At4g35100
aaaagacagatgtttatggatcgttctagccgttaccagatacgatacacgtgggatgatctgtatggattaacgaagagatctgacctgtcttttgccaaactgatgtggaaattgtgacctggatgttttagagataaagattggctgccccttgtggtaggtcagtccctttctctgaactatataatatactcaatttaatttaactagactagttgaaattgttgaagttcgctaaccataatgtacaaaaatccttcatgttccgggaaaataaatcataattaacgataggtatagtagcagcaaagataagatggtgatccattctcacatgcaatgattcggatgaagtttagccaaatattattattatccaacctcttctgcaccacgaagagactttatttacgtaatatatttgatggtccagtactactatccagacattattgttttcgtttccaaatcataaaacttgaaatcttattgattactactagggtcgatatctcactatttggaaaccgcaggctccctatttattagtttcaaactcaattgaaatgaaaatgtgtattggtggttttaatgtttgcactgcgtaatgttgcctgaactgtatatttaataaatttaccacaacttagcagatataattttgttatacgaaatcgtatagtcttcttcatggacaaagaggaaggcaatcatcattttctatgattgctcatcgacctggcatttccattaccaaaggcagcataatatcaaatcaaatatttctttgtcgtactttacttgaattaatgatattatcgtgggtcgacttaggaagtaaagttacttccatttcatatatctaaatcacatttgcgtaaaatttaagtattacacaaagtgataaagtaaattaagtaataattggctagggatattaacaaaataaataattttgattagggctacgaggataaggacaaacgtcgtattaacgtataaaaaatgtgaaaggaaaagagtaaactaagctttagatattaacactcattacgggattcctaaattgccagcacttcggattatccgtcagtccactatttatgctagtttttcttttccttgtgagcttgcactatggatttttctttatgcaattctcaatctaaatgaccattaaactttagaaaaaaatgaccattaagttttaatgtacatttgtatcaactttctaatatgccacgtccgtattgctatatacaaacataaccattcaaattaatatggctctctcaaaggaaaaaaataaaaggaatagcaaatgagtagataaattttttgatcaaaaagaataagatgcactagtattagtatatactcccatacatcatttaaccaaattttaaaattgattataatccataaatagtcttcaaatttcaacgacaaacacattaatgtgagtataaattagcatagactactatatagtttatatgcggggtgaatgtttgtctatacaagtttcgtcaaaaaaacggcaccaaccataattaatagactcttatcttttgctggtatatgaactctttttaaaaattgccgcgaatataccttcttcgtagattattaatctttttcatgtacagttgcctttagcagattatcatatgttacaaacatttgacctaatttgaaaaaatgttaccaaaaaagatttggaaaaaaagacaaagatattgagataaccgttgaagagagtttgggatgacaggctgacgagaaaaccatacacctcatttctatcattaaccctcacatggcttatctgtacatctggaccccaccaccttctacttttatctcccattttttttttcaaattattactacccaaaaaaatattcaacaaacctacaaaaacacatgtcttatttttatttatttaaacatttgtgtatttatcccattgttccatatttattagtaggaaaaatattcacattttacagaactgcaaaagatttggccatgcaaattataccccactttgattgctattttatacactatatcatacagtacaagaacaaaaatttgattgggccagtaataatgcatataactattgatgttagcaagagaaaaaaatgcattaaaacacaaatttgactattattttgcaggaacattcgtgataattttgactattattttgtcagtaataatgcatattaatcgtatgtgattttaactaaaactggaaagttccaaggaacatttagtagtttatatttttagtactaagttagtttagagggttattaactatttaatcgtactaataatgattataagcagaattatgtagactatacaaatagtacgtaataccgaaagtacccttggtttatccctggcattacaagggtttcctttttcccctttaaaatggcagaagagatacactgtcgtcgtcacacagcaccacaaactccatagctagctagagagtaaaaagaacaaagatgtcgaaagaagtgagcgaagaaggcaaaacccaccatgg

SEQ ID NO: 11 - DNA, 2421 bp, Arabidopsis thaliana
  Promoter (1)..(2421): Fragment of promoter and 5′-untranslated region of the Arabidopsis thaliana gene locus At3g04290
gtcgaccaaaagtgtaggatatataattccactctaaacacttgcatgaccaaaagtttttaagatctagggtcacgaattttggctgcaaaaggaattgaggattttgacaccaaaacaaaggaattaaggatacttcatagaacaaaaagaaaagatactcactagttacgaatgcacagaaaagtaaatataagtaattattattaattcaggtttacttctggttcggtttaaggtttataaagagtagaagtagatgatctctggcgaggtatcatttggaaaaggaaagaaacgaattcgattcagttttagactaatctagttgggttgattaaccagtgctggatcacaatgcaaaggagcttggaatcgatcttctctccagaattccattgtgttagaatgctccaattctgtttggtttaaccaaaccaaagtcgaattgattcggaaactttgttgaattttaatgagcttttcaaattcattctcatgggtcaagtttgttgatcatttgggcttgtgagatgtttgtggagtcctaacgcaagcagagagcccaaaacaaaaccatgattttccatagtggtacagtgaagtttttattttgggccattctgtttctttgatataattttatatttaaaaagtctattttcttctacagaaaaactttgtgttggttggtttttgtttacggatagacatcatttaattttactgtataactttacgaataaagacactgatatattttcacctttatggtaacgaattttatcaaaaaaaagaagaaaaaaaagtaactaattaaatagccggaaaaggttggtatgatttggagatgatattttacgttttttttttaatgtatttttcaaaaaatcaaaattcataaatcatgatgcagtcggacagggtagcttcatcatgttaatttatgacgtgagttggttgagttggtagatctcttaatttcacttctatctctctctctccaataattttatttatacagaaaattacttagtagtataaaaatcaagacaaatgaaaagtcttcttttgatcattcttgagtaattagtggaatgtattctgaattatccatttatgcatcgtaatttgttaagtcaacattttcaagtctcatgtattaacaaacataaggtgtatatgattaattaacaagtagtataattgctcttttaccaatttcatgaaaacatcaaatggtttgtgtaattttatcctagaaaatatcaagcataacccgcatttcaagaaaacaaattaaaatacattttataattacacaccaaccaaattgaacaacactcctactataaaatacatcaagattttcaaaaagtatggtattgaacacgacccattacattgacatcaatggctctaattgttatatacaccttctttttttttttgaatgcttttctctttctttctggatgctttatttatgtaatattccagctttgaaaattcgtaggtagtacgcgttaatgtgataacgttcaaaggggaggcatagttggacagaaaaagtttatccaaaaatatatatttaaccaattgaacttctcgtaattgaacatttagatggcatgagtttgacattgtataaatcttagtgaagtctagtttaaatttatccatccatcagctgatggaaacatttttagttttcaatattaaatattatgtcatgtaaaacccatgacaaaaaaaggagaggatatgattgatgagccgcctaaattcacgtatgttacgttaactatatagcatatagatatttcacatattaaccaagtagttcgaaatggatgggttatacaatatggtttctttggggaccgtccggtcactgcatctcgatttgtgacattggatataaacacaaaactacacaaaaatggagatgaagaacggtaaagattagtttctttggatcgatatacatatacctaatcacttcaaaatttacacgtagattgcagacattgagatcagtaaccgtcaatcaaaaaaattatgtcacatcgaatttcaatggttgaaagataattccattttaggagaacaagcagtagtacaatttctaacatttattacatccaaacaacaaacaagaccaatttttatggtgtatcagttggcagcctctattaatgtctctattttcttaaaatcacaccaacaacgaaaaaaaaaaaaaaaaaaaaacagatacacattacttattgaaccctaaacctattgtcagtgtatatatataacacacactctccatatgctctcatatcatacgtcaatttataaacgtttcttctcattcgtcatctagtcccattctctgtccataacaatgaatatcaattgttctccccatgg

SEQ ID NO: 12 - DNA, 2345 bp, Arabidopsis thaliana
  Promoter (1)..(2345): Fragment of promoter and 5′-untranslated region of the Arabidopsis thaliana gene locus At5g46110
cagtaggaggcaaaccaatctctctcatattcttgtaaaacttaaatgccaaatttaactgattctcttccacgagaatggcaagaacagtgacataagccttctggctaggatcacaatcaaaatccttcattttgtgaaaaacccttaaagaatcaaacggcctatgaacccgaccataccctcgacatatcgaaagcaatatatcttcactaacaacacaattctcaatcttcattctaacaatcagatcttcagctgctttgaacttattagcagagaccaacctcaagaccatataaccaaaagaactctgatcatgtacgtacccatttgcgtattccgccgttgcggaatcaaaaactgccatagacttctccacatctttttcagctcgcatcagtttgataacctgtgaaggcgtgatgttctttgaccacttgaacatcatcactttgctacccattactaaacaatgataccttccaacaatagcaaagaatacacctttttatagagaagaatctcagctacgcattacgtcgaacaggttgtgtgcataaacgatttcgataggctcaaaccaaatgaaagaaacacagaccagaaaaatcatttgatcttcaaaacatacgagttccaaaagtgaaggaagcaacaatgaaactcgttacgaactagaaggttaatcaaattgccggagaagaatcgctcaccagttttggctagggtttatgaaatgggagactttagctgcaaagagaagagtctctggacgatttagagggtgtctctctaacaggcaacaaagtacatattattacagtattaaccaaatttaaacgaattaagtgtcaacaaaagcttatataaaaaatttaaagtttaaaaattataaaatatgtcaacaatattttagtacttaaaattattatgcgaaatatttagatcaatggactactcatctaatatatttgcacctaattttaaagtataaattcaaccaataattagaaaatgatagcttatactcaaattcaacaaattatatataaatgtatagatactacaatatcattaacaaaagtcaccttaaataaatacacatatcttttatgttctctattgttttgcgtacgctaacacaatttctcatatgcaaaaggatgaatgagtaacaaattacctcataagaacaatcatctttgcttacatactaatacaataatcactcaatcaaccaataacatcaatcacataggtttacatacaataatcactcaatcaacttcataagaagaatcatgtttacttaattcatcaattatccccaaaaacactactattaagtataaactataacatatttgtagtgatgggtcaacatttttatcatatttaaactcgggtttcctcaaatcgagaaatagtgaacatgtaatattaattttaaatcgcaattacagaaattaattgaatttggtcaaatggacagaattttataaattgggtggaactagaaaaaaaaaaaaaaagagtatagggtgaattgagtacatgaaagtacatggtaatcctagttaaacgcataatacatgtgggttcatttgtatttttttgtaacttacgagtaaactggctacaacaaaaaaaaattagaagatttttttgttttgtagaaaaccctaattttagttatagttgtataactttgataaaattataaaattgtattacgaaaaaagtaataagatattcaaaaaagcctagaataacgtatatgactatgagcatgaaactgcaagtcaaatgctgacagacaaccataaacaaaagaaattaaatagagatacctttaaaataagtaaaatttcatttataaaaaatctactttcttgtgaatctgtcacgttcaataatttgaagaccactcaacatacaaggtaaataatgaaaaataaaatctaccaaaatttcaattattattatcttccaaaaaaacaaaattatacagatgatgatggtgatatggaacttcgattggctaatattcactgtgtctctaaaaaccatccacttatcaagataagatggaccctacactcatccaatctaaaccagtatctcaagattcttatctaattacatcattctctaccgttagatgaaattgaccattaaccctaccataactccatacaccgcgagatactggattaaccaaatcgagatcatcgtagccgtccgatcaacaagtaccatctcttgaaatactcgaaatcctcataagtccgtccctctttgctctcactatcaaaactctgaatttcgatttcaatggagtc

SEQ ID NO: 13 - DNA, 25 bp, Artificial sequence
  Oligonucleotide primer M1as
tgtttatagatttaaaatggtaata

SEQ ID NO: 14 - DNA, 26 bp, Artificial sequence
  Oligonucleotide primer M1s
gattttgtaaggactaagaagaaata

SEQ ID NO: 15 - DNA, 22 bp, Artificial sequence
  Oligonucleotide primer M1ss
taatgataatttcgtttcaata

SEQ ID NO: 16 - DNA, 34 bp, Artificial sequence
  Oligonucleotide primer M1svl
gtcgacaggaaattttagtttcttttaagtgtgc

SEQ ID NO: 17 - DNA, 28 bp, Artificial sequence
  Oligonucleotide primer M2as
tttcgatgaagcttctatggatatattt

SEQ ID NO: 18 - DNA, 28 bp, Artificial sequence
  Oligonucleotide primer M2s
cattgattgggaagactattttatgata

SEQ ID NO: 19 - DNA, 28 bp, Artificial sequence
  Oligonucleotide primer M2ss
aaaatgacacaaatgtaatggacaagta

SEQ ID NO: 20 - DNA, 27 bp, Artificial sequence
  Oligonucleotide primer M2svl
gtcgacgatatggtcgtaaacgcaatg

SEQ ID NO: 21 - DNA, 34 bp, Artificial sequence
  Oligonucleotide primer M3as
cccggggttttcacagaaatttgagagaagaaag

SEQ ID NO: 22 - DNA, 28 bp, Artificial sequence
  Oligonucleotide primer M3s
attttcagaaattgttatgtaacttcta

SEQ ID NO: 23 - DNA, 28 bp, Artificial sequence
  Oligonucleotide primer M3ss
aaagtttgatgttcggaaagagtttaga

SEQ ID NO: 24 - DNA, 36 bp, Artificial sequence
  Oligonucleotide primer M3svl
gtcgacgattttatatataacagagctagtacaacg

SEQ ID NO: 25 - DNA, 16 bp, Artificial sequence
  Oligonucleotide primer M4as
ccatggtgggttttgc

SEQ ID NO: 26 - DNA, 22 bp, Artificial sequence
  Oligonucleotide primer M4s
ctgcagaaaagacagatgttta

SEQ ID NO: 27 - DNA, 22 bp, Artificial sequence
  Oligonucleotide primer M5as
gtcgaccaaaagtgtaggatat

SEQ ID NO: 28 - DNA, 22 bp, Artificial sequence
  Oligonucleotide primer M5s
ccatggggagaacaattgatat

SEQ ID NO: 29 - DNA, 22 bp, Artificial sequence
  Oligonucleotide primer M6as
ccatgggactccattgaaatcg

SEQ ID NO: 30 - DNA, 19 bp, Artificial sequence
  Oligonucleotide primer M6s
ctgcagtaggaggcaaacc

SEQ ID NO: 31 - DNA, 15 bp, Artificial sequence
  Degenerate random primers
ntcgastwtsgwgtt

SEQ ID NO: 32 - DNA, 16 bp, Artificial sequence
  Degenerate random primers
ngtcgaswganawgaa

SEQ ID NO: 33 - DNA, 16 bp, Artificial sequence
  Degenerate random primers
wcagntgwtngtnctg

(19) United States
(12) Patent Application Publication
(10) Pub. No.: US 2009/0068163 A1
(43) Pub. Date: Mar. 12, 2009

(54) Transgenic expression cartridges for expressing nucleic acids in the flower tissue of plants

(75) Inventors: Martin Klebsattel, Karlsruhe (DE); Ralf Flachmann, Quedlinburg (DE); George Mather Sauer, Quedlinburg (DE); Christel Renate Schopfer, Quedlinburg (DE)

Correspondence Address: CONNOLLY BOVE LODGE & HUTZ, LLP, P O BOX 2207, WILMINGTON, DE, 19899, US

(73) Assignee: SunGene GmbH, Gatersleben (DE)

(21) Appl. No.: 11/919,862

(22) PCT Filed: May 3, 2006
(86) PCT No.: PCT/EP2006/062012

§ 371 Date: Oct. 31, 2007

(30) Foreign Application Priority Data
May 4, 2005 (DE) 10 2005 021 365.0

(51) Int. Cl. A61K 8/43; C12N 15/82; C12Q 1/68; A01H 5/00; A61K 31/70; A61K 31/20; C12N 1/21; C12N 1/19; C12N 1/15

(52) U.S. Cl. 424/94.1; 435/468; 435/6; 435/320.1; 800/295; 435/252.3; 435/254.2; 435/254.11; 435/325; 800/13; 514/23; 514/558

(57) ABSTRACT

The invention relates to methods for the targeted transgenic expression of nucleic acid sequences in tissues of plants, and to transgenic expression cassettes and expression vectors comprising promoters with expression specificity for floral tissues. The invention further relates to organisms (preferably plants) transformed with these transgenic expression cassettes or expression vectors, to cultures, parts or propagation material derived from these organisms, and to their use for the production of foodstuffs, feedstuffs, seed, pharmaceuticals or fine chemicals.

The invention relates to methods for the targeted transgenic expression of nucleic acid sequences in tissues of plants, and to transgenic expression cassettes and expression vectors comprising promoters with expression specificity for floral tissues. The invention further relates to organisms (preferably plants) transformed with these transgenic expression cassettes or expression vectors, to cultures, parts or propagation material derived from these organisms, and to their use for the production of foodstuffs, feedstuffs, seed, pharmaceuticals or fine chemicals.

The aim of biotechnological operations on plants is to produce plants with advantageous novel properties, for example for increasing the agricultural productivity, for increasing the quality of foodstuffs or for producing particular chemicals or pharmaceuticals (Dunwell J M (2000) J Exp Bot 51 Spec No: 487-96). A basic precondition for transgenic expression of particular genes is the provision of plant-specific promoters. Promoters are important tools in plant biotechnology for controlling the expression of particular genes in a transgenic plant and thus achieving particular traits of the plant.

Various plant promoters are known, for example constitutive promoters such as the promoter of the agrobacterium nopaline synthase, the TR double promoter or the promoter of the cauliflower mosaic virus (CaMV) 35S transcript (Odell et al. (1985) Nature 313:810-812). A disadvantage of these promoters is that they are constitutively active in virtually all tissues of the plant. Targeted expression of genes in particular plant parts or at particular times of development is not possible with these promoters.

Promoters having specificities for various plant tissues such as anthers, ovaries, flowers, leaves, stalks, roots, tubers or seeds have been described. The stringency of the specificity and the expression activity of these promoters varies widely.

The flower of plants serves for sexual reproduction of flowering plants. The flowers of plants—especially the petals—frequently accumulate large amounts of secondary plant products such as, for example, terpenes, anthocyans, carotenoids, alkaloids and phenylpropanoids, which serve as scents, defensive substances or as colorants in the flower. Many of these substances are of commercial interest. In addition, the flower bud and the flower of the plant is a sensitive organ, especially to stress factors such as cold.

The Arabidopsis thaliana gene locus At5g33370 (derived protein GenBank Acc.-No.: NP_{—}198322) codes for a putative GDSL-motif lipase/hydrolase family protein.

The Arabidopsis thaliana gene locus At5g22430 (derived protein GenBank Acc.-No.: NP_{—}568418) codes for an expressed protein. The Arabidopsis thaliana gene locus At1g26630 (derived protein GenBank Acc.-No.: NP_{—}173985) codes for a putative eukaryotic translation initiation factor 5A/eIF-5. The Arabidopsis thaliana gene locus At4g35100 (derived protein GenBank Acc.-No.: NP_{—}195236) codes for a putative plasma membrane intrinsic protein (SIMIP). The Arabidopsis thaliana gene locus At3g04290 (derived protein GenBank Acc.-No.: NP_{—}187079) codes for a putative GDSL-motif lipase/hydrolase family protein. The Arabidopsis thaliana gene locus At5g46110 (derived protein GenBank Acc.-No.: NP_{—}568655) codes for a putative phosphate/triose-phosphate translocator.

The function, transcription pattern and expression pattern of these genes have not been described.

Flower-specific promoters such as, for example, the phytoene synthase promoter (WO 92/16635), the promoter of the P-rr gene (WO 98/22593) or the promoter of the APETALA3 gene (Hill T A et al. (1998) Development 125:1711-1721) are known. However, all these promoters have one or more disadvantages which are prejudicial to wide use:
1) within the flower they are specific for one or more floral tissues and do not guarantee expression in all floral tissues.
2) they are—as in the example of the APETALA3 gene which is involved in floral development—highly regulated during floral development and are not active in all phases of floral development.
3) they occasionally show strong secondary activities in other plant tissues.

It is an object of the present invention to provide methods and suitable promoters for the targeted transgenic expression of nucleic acids in floral tissues. We have found that this object is achieved in particular by providing promoters of genes with the gene locus names At5g33370, At5g22430, At1g26630, At4g35100, At3g04290 and At5g46110.

These promoters show an expression in all flower organs. This expression pattern can be observed in the flower bud, the flower and the senescent flower.

A first aspect of the invention relates to methods for the targeted, transgenic expression of nucleic acid sequences in the floral tissues of plants, including the following steps:
I. introduction of a transgenic expression cassette into plant cells, where the transgenic expression cassette comprises at least the following elements
   a) at least one promoter sequence selected from the group of sequences consisting of
      i) the promoter sequences as shown in SEQ ID NO: 1, SEQ ID NO: 4, SEQ ID NO: 7, SEQ ID NO: 10, SEQ ID NO: 11 and SEQ ID NO: 12 and
      ii) functional equivalents of the promoter sequences as shown in SEQ ID NO: 2, SEQ ID NO: 3, SEQ ID NO: 5, SEQ ID NO: 6, SEQ ID NO: 8 and SEQ ID NO: 9 with essentially the same promoter activity as a promoter as shown in SEQ ID NO: 1, SEQ ID NO: 4, SEQ ID NO: 7, SEQ ID NO: 10, SEQ ID NO: 11 and SEQ ID NO: 12 and
      iii) functionally equivalent fragments of the sequences under i) or ii) with essentially the same promoter activity as a promoter sequence as shown in i) or ii) and
   b) at least one further nucleic acid sequence, and
   c) where appropriate further genetic control elements,
where at least one promoter sequence and one further nucleic acid sequence are functionally linked with one another, and the further nucleic acid sequence is heterologous in relation to the promoter sequence, and
II. selection of transgenic cells which comprise said expression cassette stably integrated into the genome, and
III. regeneration of complete plants from said transgenic cells, where at least one of the further nucleic acid sequence is expressed in essentially all of the floral tissues.

A further aspect relates to transgenic expression cassettes as can be employed in the method of the invention. The transgenic expression cassettes preferably comprise for the targeted, transgenic expression of nucleic acid sequences in floral tissues of plants
a) at least one promoter sequence selected from the group of sequences consisting of
   i) the promoter sequences as shown in SEQ ID NO: 1, SEQ ID NO: 4, SEQ ID NO: 7, SEQ ID NO: 10, SEQ ID NO: 11 and SEQ ID NO: 12 and
   ii) functional equivalents of the promoter sequences as shown in SEQ ID NO: 2, SEQ ID NO: 3, SEQ ID NO: 5, SEQ ID NO: 6, SEQ ID NO: 8 and SEQ ID NO: 9 with essentially the same promoter activity as a promoter as shown in SEQ ID NO: 1, SEQ ID NO: 4, SEQ ID NO: 7, SEQ ID NO: 10, SEQ ID NO: 11 and SEQ ID NO: 12 and
   iii) functionally equivalent fragments of the sequences under i) or ii) with essentially the same promoter activity as a promoter sequence as shown in i) or ii)
      and
b) at least one further nucleic acid sequence, and
c) where appropriate further genetic control elements,
   where at least one promoter sequence and one further nucleic acid sequence are functionally linked with one another, and the further nucleic acid sequence is heterologous in relation to the promoter sequence.

The expression cassettes of the invention may comprise further genetic control sequences and/or additional functional elements.

It is possible and preferred for the transgenic expression cassettes to make possible, through the nucleic acid sequence to be expressed transgenically, the expression of a protein encoded by said nucleic acid sequence and/or the expression of a sense-RNA, antisense-RNA or double-stranded RNA encoded by said nucleic acid sequence.

The transgenic expression cassettes according to the invention are particularly advantageous since they allow a selective expression in the tissues of the flower bud and of the flower of the plant and make possible a large number of uses, such as, for example, a resistance to stress factors such as cold or a targeted synthesis of secondary plant constituents. The expression is essentially constant over the entire development period of the flower bud and flower.

The transgenic expression cassettes according to the invention, the transgenic expression vectors and transgenic organisms derived therefrom may comprise functional equivalents of the promoter sequences described under SEQ ID NO: 1, 2, 3, 4, 5, 6, 7, 8, 9, 10, 11 and 12.

A further aspect of the invention relates to transgenic expression vectors which comprise one of the expression cassettes of the invention.

A further aspect of the invention relates to transgenic organisms which comprise one of the expression cassettes or expression vectors of the invention. The organism can be selected from the group consisting of bacteria, yeasts, fungi, nonhuman, animal and plant organisms or cells, cell cultures, parts, tissues, organs or propagation material derived therefrom, and the organism is preferably selected from the group of the agricultural crop plants.

A further aspect of the invention relates to the use of said organisms or cells, cell cultures, parts, tissues, organs or propagation material derived therefrom to produce foodstuffs, feedstuffs, seeds, pharmaceuticals or fine chemicals, where the fine chemicals are preferably enzymes, vitamins, amino acids, sugars, saturated or unsaturated fatty acids, natural or synthetic flavorings, aromatizing substances or colorants. The invention further includes methods for producing said foodstuffs, feedstuffs, seeds, pharmaceuticals or fine chemicals employing the organisms of the invention or cells, cell cultures, parts, tissues, organs or propagation material derived therefrom.

The promoter activity of a functionally equivalent promoter is referred to as “substantially the same” when the transcription of a particular nucleic acid sequence to be expressed transgenically under the control of said functionally equivalent promoter shows a targeted expression in essentially all floral tissues under conditions which are otherwise unchanged.

“Flower” generally means a shoot of limited growth whose leaves have been transformed into reproductive organs. The flower consists of various “floral tissues” such as, for example, the sepals, the petals, the stamens or the carpels. Androeceum is the term used for the totality of stamens in the flower. The stamens are located within the circle of petals and sepals. A stamen is composed of a filament and of an anther located at the end. The latter in turn is divided into two thecae which are connected together by a connective. Each theca consists of two pollen sacs in which the pollen is formed.

In relation to the floral tissues, “essentially all floral tissues” means that some of these tissues, in total or at certain points in time of their development, may lack substantial expression, the percentage of these tissues of the total weight of the floral tissues being, however, preferably less than 20% by weight, preferably less than 10% by weight, especially preferably less than 5% by weight, very especially preferably less than 1% by weight.

“Targeted” means in relation to expression in the floral tissues of plants preferably that the expression under the control of one of the promoters of the invention in the floral tissues is preferably at least twice, very especially preferably at least ten times, most preferably at least one hundred times that in a non-floral tissue such as, for example, the leaves.

That promoters according to the invention “essentially lack expression in the pollen and ovaries” preferably means that the statistical mean of the expression over all reproductive floral tissues amounts to no more than 10%, preferably no more than 5%, most preferably no more than 1% of the statistical mean of the expression over all floral tissues under the same conditions.

Expression is preferably essentially constant within the floral tissues. In this context, “essentially constant” preferably means that the standard deviation of the expression between the individual floral tissues based on the statistical mean of the expression over all floral tissues is less than 50%, preferably 20%, especially preferably 10%, very especially preferably 5%.

Preferably, expression within at least one particular floral tissue is essentially constant over all developmental stages of the flower. In this context, “essentially constant” preferably means that the standard deviation of the expression between the individual points in time of the development of the respective floral tissue based on the statistical mean of the expression over all points in time of development is less than 50%, preferably 20%, especially preferably 10%, very especially preferably 5%.

The nucleic acid sequences preferably employed for estimating the level of expression are those which are functionally linked to the promoter to be tested and code for easily quantifiable proteins. Very particular preference is given in this connection to reporter proteins (Schenborn E, Groskreutz D. (1999) Mol Biotechnol 13(1): 29-44) such as the green fluorescence protein (GFP) (Chui W L et al. (1996) Curr Biol 6:325-330; Leffel S M et al. (1997) Biotechniques 23(5):912-8), chloramphenicol transferase, luciferase (Millar et al. (1992) Plant Mol Biol Rep 10:324-414), β-glucuronidase or b-galactosidase. Very particular preference is given to β-glucuronidase (Jefferson et al. (1987) EMBO J 6:3901-3907).

“Conditions which are otherwise unchanged” means that the expression initiated by one of the transgenic expression cassettes to be compared is not modified by combination with additional genetic control sequences, for example enhancer sequences. Unchanged conditions further means that all general conditions such as, for example, plant species, stage of development of the plants, culture conditions, assay conditions (such as buffer, temperature, substrates etc.) are kept identical between the expressions to be compared.

“Transgenic means—for example in relation to an expression cassette, or to an expression vector or transgenic organism comprising it—all those constructs which result from genetic engineering methods and in which either
a) the promoter as shown in SEQ ID NO: 1, 4, 7, 10, 11 and 12 or a functional equivalent thereof or a part of the above, or
b) a further nucleic acid sequence which is functionally linked with a), or
c) (a) and (b)
   are not located in their natural genetic environment or have been modified by genetic engineering methods, it being possible for the modification to be, for example, substitutions, additions, deletions, inversion or insertions of one or more nucleotide residues. The promoter sequence according to the invention which is present in the expression cassettes (for example the sequence as shown in SEQ ID NO: 1, 2, 3, 4, 5, 6, 7, 8, 9, 10, 11 or 12) is preferably heterologous in relation to the further nucleic acid sequence which is to be expressed transgenically and is functionally linked thereto. “Heterologous” means in this connection that the further nucleic acid sequence does not code for the gene which is naturally under the control of said promoter.

“Natural genetic environment” means the natural chromosomal locus in the original organism or the presence in a genomic library. In the case of a genomic library, the natural genetic environment of the nucleic acid sequence is preferably still retained at least in part. The environment flanks the nucleic acid sequence at least on one side and has a sequence length of at least 50 bp, preferably at least 500 bp, particularly preferably at least 1000 bp, very particularly preferably at least 5000 bp. A naturally occurring expression cassette—for example the naturally occurring combination of the promoter of a gene coding for a protein in accordance with the genes with the gene locus names At5g33370, At5g22430, At1g26630, At4g35100, At3g04290 and At5g46110 or a functional equivalent of these with its corresponding coding sequences—becomes a trangenic expression construct when the latter is modified by non-natural, synthetic (“artificial”) methods such as, for example, a mutagenesis. Appropriate methods are described (U.S. Pat. No. 5,565,350; WO 00/15815; see also above). “Transgenic” means in relation to an expression (“transgenic expression”) preferably all those expressions caused by use of a transgenic expression cassette, transgenic expression vector or transgenic organism—complying with the definitions given above.

“Functional equivalents” of a promoter as shown in SEQ ID NO: 1, 4, 7, 10, 11 and 12 means, in particular, natural or artificial mutations of a promoter, for example as shown in SEQ ID NO: 2, 3, 5, 6, 8, and 9, and homologous sequences from other organisms, preferably from plant organisms, which have essentially the same promoter activity as one of the promoters as shown in SEQ ID NO: 1, 4, 7, 10, 11 or 12.

Functional equivalents also comprise all those sequence which are derived from the complementary counterstrand of the sequences defined by SEQ ID NO: 1, 2, 3, 4, 5, 6, 7, 8, 9, 10, 11 or 12 and which have essentially the same promoter activity.

Functional equivalents to the promoters as shown in 1, 2, 3, 4, 5, 6, 7, 8, 9, 10, 11 or 12 preferably comprise those sequences which
a) have essentially the same promoter activity as one of the promoters as shown in SEQ ID NO: 1, 2, 3, 4, 5, 6, 7, 8, 9, 10, 11 or 12, and
b) have a homology of at least 50%, preferably 70%, more preferably at least 80%, particularly preferably at least 90%, very particularly preferably at least 95%, most preferably 99%, with the sequence of one of the promoters as shown in SEQ ID NO: 1, 2, 3, 4, 5, 6, 7, 8, 9, 10, 11, or 12, where the homology extends over a length of at least 100 base pairs, preferably at least 200 base pairs, particularly preferably of at least 300 base pairs, very particularly preferably of at least 400 base pairs, most preferably of at least 500 base pairs.

It is possible in this connection for the level of expression of the functional equivalents to differ both downwards and upwards from a comparison value. Preference is given in this connection to the sequences whose level of expression, measured on the basis of the transcribed mRNA or the subsequently translated protein, under conditions which are otherwise unchanged differs quantitatively by not more than 50%, preferably 25%, particularly preferably 10%, from a comparison value obtained with the promoters described by SEQ ID NO: 1, 2, 3, 4, 5, 6, 7, 8, 9, 10, 11 or 12. Particularly preferred sequences are those whose level of expression, measured on the basis of the transcribed mRNA or the subsequently translated protein, under conditions which are otherwise unchanged exceeds quantitatively by more than 50%, preferably 100%, particularly preferably 500%, very particularly preferably 1000%, a comparison value obtained with the promoter described by SEQ ID NO: 1, 2, 3, 4, 5, 6, 7, 8, 9, 10, 11 or 12.

Examples of promoter sequences employed in the transgenic expression cassettes or transgenic expression vectors of the invention can easily be found for example in further organisms whose genomic sequence is known, such as, for example, Arabidopsis thaliana, Brassica napus, Nicotiana tabacum, Solanum tuberosum, Helianthium annuus, Linum sativum, by homology comparisons in databases. A possible and preferred starting point for this is the coding regions of the gene whose promoters are described by, for example, SEQ ID NO: 1, 4, 7, 10, 11 or 12. Starting from, for example, the cDNA sequences the sequences of the genes with the gene locus names At5g33370, At5g22430, At1g26630, At4g35100, At3g04290 and At5g46110 it is possible easily to identify, in a manner familiar to the skilled worker, the corresponding homologous genes in other plant species by screening databases or gene libraries (using appropriate gene probes).

In a preferred embodiment of the invention, functional equivalents of the promoters described by SEQ ID NO: 1, 4, 7, 10, 11 and 12 comprise all those promoters which are located in a plant organism in the 5′-direction upstream of a genomic sequence which codes for a protein with at least 60%, preferably at least 80%, especially preferably at least 90%, most preferably at least 95% homology. Preferably, these take the form of the genes with the gene locus names At5g33370, At5g22430, At1g26630, At4g35100, At3g04290 and At5g46110 corresponding to the proteins with the sequences of Acc. No. NP_{—}198322, NP_{—}568418, NP_{—}173985, NP_{—}195236, NP_{—}187079, NP_{—}568655, where said promoters constitute the natural promoter of said genomic sequence.

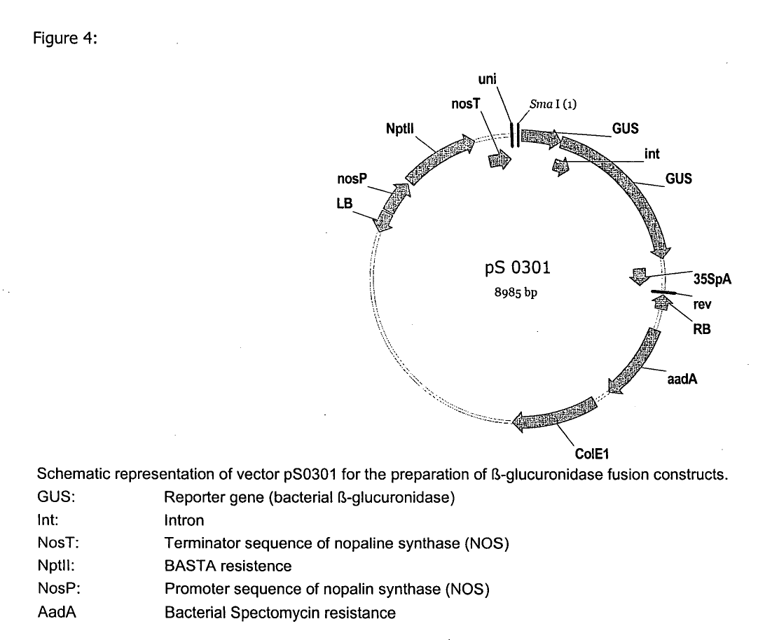

Various methods for identifying and isolating, starting from a nucleic acid sequence (e.g. a gene transcript such as, for example, a cDNA), the promoter of the corresponding gene are known to the skilled worker. In principle, all methods for amplifying flanking chromosomal sequences are available for example for this purpose. The two most commonly used methods are inverse PCR (“iPCR”; diagrammatically depicted in FIG. 10) and “thermal asymmetric interlaced PCR” (“TAIL PCR”).

For the iPCR, genomic DNA of the organism from which the functionally equivalent promoter is to be isolated is completely digested with a given restriction enzyme, and then the individual fragments are religated, i.e. linked to themselves to give a circular molecule, in a diluted mixture. The large number of resulting circular DNA molecules also includes those comprising the known sequence (for example the sequence coding for the homologous protein). Starting from this, the circular molecule can be amplified by PCR using a primer pair where both primers are able to anneal to the known sequence segment. One possible embodiment of the iPCR is reproduced in example 4.

The TAIL-PCR is based on the use of firstly a set of successively truncated highly specific primers which anneal to the known genomic sequence (for example the sequence coding for the homologous protein), and secondly a set of shorter random primers with a lower melting temperature, so that a less sequence-specific annealing to genomic DNA flanking the known genomic sequence takes place. Annealing of the primers to the DNA to be amplified is possible with such a primer combination to make specific amplification of the desired target sequence possible. One possible embodiment of the TAIL-PCR is reproduced for example in example 4.

A further aspect of the invention relates to methods of preparing a transgenic expression cassette with specificity for floral tissues, comprising the following steps:
I. isolation of a promoter with specificity for floral tissue, where at least one nucleic acid sequence or a part thereof is employed in the isolation, where said nucleic acid sequence codes for an amino acid sequence which comprises at least some of the sequences of Acc No. NP_{—}198322, NP_{—}568418, NP_{—}173985, NP_{—}195236, NP_{—}187079 or NP_{—}568655.
II. functional linkage of said promoter with a further nucleic acid sequence, where said nucleic acid sequence is heterologous in relation to the promoter.

Said nucleic acid sequence preferably codes for an amino acid sequence comprising a sequence comprising sequences of Acc. No. NP_{—}198322, NP_{—}568418, NP_{—}173985, NP_{—}195236, NP_{—}187079 or NP_{—}568655.

“Part” means in relation to the nucleic acid sequence preferably a sequence of at least 10 bases, preferably 15 bases, particularly preferably 20 bases, most preferably 30 bases. In a preferred embodiment, the method of the invention is based on the polymerase chain reaction, where said nucleic acid sequence or a part thereof is employed as primer. Methods known to the skilled worker, such as, for example, ligation etc., can be employed for the functional linkage (see below).

“Mutation” means substitution, addition, deletion, inversion or insertion of one or more nucleotide residues. Thus, for example, the present invention also comprises those nucleotide sequences which are obtained by modification of the promoters as shown in SEQ ID NO: 1, 2, 3, 4, 5, 6, 7, 8, 9, 10, 11 or 12. The purpose of such a modification may be the further delimitation of the sequence comprised therein or else, for example, the insertion of further restriction enzyme cleavage sites, the removal of excess DNA or the addition of further sequences, for example further regulatory sequences.

Where insertions, deletions or substitutions, such as, for example, transitions and transversions, are appropriate, it is possible to use techniques known per se, such as in vitro mutagenesis, primer repair, restriction or ligation. Transition means a base-pair exchange of a purine/pyrimidine pair into another purine/pyrimidine pair (e.g. A-T for G-C). Transversion means a base-pair exchange of a purine/pyrimidine pair for a pyrimidine/purine pair (e.g. A-T for T-A). Deletion means removal of one or more base pairs. Insertion means introduction of one or more base pairs.

Complementary ends of the fragments for ligation can be made available by manipulations such as, for example, restriction, chewing back or filling in of overhangs for blunt ends. Analogous results are also obtainable by using the polymerase chain reaction (PCR) using specific oligonucleotide primers.

Identity between two nucleic acids means the identity of the nucleotides over the complete nucleic acid length in each case, in particular the identity which is calculated by comparison with the aid of the Vector NTI Suite 7.1 software from Informax (USA) using the Clustal method (Higgins D G, Sharp P M. Fast and sensitive multiple sequence alignments on a microcomputer. Comput Appl. Biosci. 1989 April; 5(2):151-1), setting the following parameters:

### Multiple Alignment Parameter:

| | |
|---|---|
| Gap opening penalty | 10 |
| Gap extension penalty | 10 |
| Gap separation penalty range | 8 |
| Gap separation penalty | off |
| % identity for alignment delay | 40 |
| Residue specific gaps | off |
| Hydrophilic residue gap | off |
| Transition weighing | 0 |

### Pairwise Alignment Parameter:

| FAST algorithm | on |
|---|---|
| K-tuple size | 1 |
| Gap penalty | 3 |
| Window size | 5 |
| Number of best diagonals | 5 |

For example, a sequence having a homology of at least 50% based on nucleic acid, for example with the sequence SEQ ID NO: 1, is understood as meaning a sequence which, on comparison with the sequence SEQ ID NO: 1 in accordance with the above program algorithm with the above parameter set, has a homology of at least 50%.

Identity between two proteins is understood as meaning the identity of the amino acids over in each case the entire protein length, in particular the identity which is calculated by comparison with the aid of the Vector NTI Suite 7.1 software from Informax (USA) using the Clustal method (Higgins D G, Sharp P M. Fast and sensitive multiple sequence alignments on a microcomputer. Comput Appl. Biosci. 1989 April; 5(2):151-1), setting the following parameters:

### Multiple Alignment Parameter:

| | |
|---|---|
| Gap opening penalty | 10 |
| Gap extension penalty | 10 |
| Gap separation penalty range | 8 |
| Gap separation penalty | off |
| % identity for alignment delay | 40 |
| Residue specific gaps | off |
| Hydrophilic residue gap | off |
| Transition weighing | 0 |

### Pairwise Alignment Parameter:

| FAST algorithm | on |
|---|---|
| K-tuple size | 1 |
| Gap penalty | 3 |
| Window size | 5 |
| Number of best diagonals | 5 |

Homology between two polypeptides means the identity of the amino acid sequence over the respective sequence length, which is calculated by comparison with the aid of the GAP program algorithm (Wisconsin Package Version 10.0, University of Wisconsin, Genetics Computer Group (GCG), Madison, USA), setting the following parameters:

| | |
|---|---|
| Gap Weight: 8 | Length Weight: 2 |
| Average Match: 2.912 | Average Mismatch: −2.003 |

For example, a sequence having a homology of at least 60% based on protein with the sequences of NP_{—}198322, NP_{—}568418, NP_{—}173985, NP_{—}195236, NP_{—}187079, NP_{—}568655 means a sequence which has a homology of at least 60% on comparison by the above program algorithm with the above set of parameters.

Functional equivalents also means DNA sequences which hybridize under standard conditions with one of the nucleic acid sequence coding for one of the promoters as shown in SEQ ID NO: 1, 2, 3, 4, 5, 6, 7, 8, 9, 10, 11 or 12, or with the nucleic acid sequences complementary thereto, and which have substantially the same promoter properties.

The term standard hybridization conditions is to be understood broadly and means both stringent and less stringent hybridization conditions. Such hybridization conditions are described inter alia in Sambrook J, Fritsch E F, Maniatis T et al., in Molecular Cloning—A Laboratory Manual, 2^{nd} edition, Cold Spring Harbor Laboratory Press, 1989, pages 9.31-9.57 or in Current Protocols in Molecular Biology, John Wiley & Sons, N.Y. (1989), 6.3.1-6.3.6. For example, the conditions during the washing step can be selected from the range of conditions limited by those of low stringency (with approximately 2×SSC at 50° C.) and those of high stringency (with approximately 0.2×SSC at 50° C., preferably at 65° C.) (20×SSC: 0.3 M sodium citrate, 3 M NaCl, pH 7.0). In addition, the temperature during the washing step can be raised from low-stringency conditions at room temperature, approximately 22° C., to more stringent conditions at approximately 65° C. Both parameters, the salt concentration and the temperature, can be varied simultaneously, and it is also possible for one of the two parameters to be kept constant and only the other to be varied. It is also possible to employ denaturing agents such as, for example, formamide or SDS during the hybridization. Hybridization in the presence of 50% formamide is preferably carried out at 42° C. Some exemplary conditions for hybridization and washing step are given below:

(1) Hybridization Conditions with for Example
a) 4×SSC at 65° C., or
b) 6×SSC, 0.5% SDS, 100 μg/ml denatured fragmented salmon sperm DNA at 65° C., or
c) 4×SSC, 50% formamide, at 42° C., or
d) 2× or 4×SSC at 50° C. (low-stringency condition), or
e) 2× or 4×SSC, 30 to 40% formamide at 42° C. (low-stringency condition), or
f) 6×SSC at 45° C., or,
g) 0.05 M sodium phosphate buffer pH 7.0, 2 mM EDTA, 1% BSA and 7% SDS.
   (2) Washing Steps with for Example
a) 0.1×SSC at 65° C., or
b) 0.1×SSC, 0.5% SDS at 68° C., or
c) 0.1×SSC, 0.5% SDS, 50% formamide at 42° C., or
d) 0.2×SSC, 0.1% SDS at 42° C., or
e) 2×SSC at 65° C. (low-stringency condition), or
f) 40 mM sodium phosphate buffer pH 7.0, 1% SDS, 2 mM EDTA.

Methods for preparing functional equivalents of the invention preferably comprise the introduction of mutations into one of the promoters as shown in SEQ ID NO: 1, 2, 3, 4, 5, 6, 7, 8, 9, 10, 11 or 12. Mutagenesis may be random, in which case the mutagenized sequences are subsequently screened for their properties by a trial and error procedure. Particularly advantageous selection criteria include for example the level of the resulting expression of the introduced nucleic acid sequence in a floral tissue.

Methods for mutagenesis of nucleic acid sequences are known to the skilled worker and include by way of example the use of oligonucleotides with one or more mutations compared with the region to be mutated (e.g. in a site-specific mutagenesis). Primers with approximately 15 to approximately 75 nucleotides or more are typically employed, with preferably about 10 to about 25 or more nucleotide residues being located on both sides of the sequence to be modified. Details and procedure for said mutagenesis methods are familiar to the skilled worker (Kunkel et al. (1987) Methods Enzymol 154:367-382; Tomic et al. (1990) Nucl Acids Res 12:1656; Upender et al. (1995) Biotechniques 18(1):29-30; U.S. Pat. No. 4,237,224). A mutagenesis can also be achieved by treating for example transgenic expression vectors comprising one of the nucleic acid sequences of the invention with mutagenizing agents such as hydroxylamine.

An alternative possibility is to delete nonessential sequences of a promoter of the invention without significantly impairing the essential properties mentioned. Such deletion variants represent functional equivalents to the promoters described by SEQ ID NO: 1, 2, 3, 4, 5, 6, 7, 8, 9, 10, 11 or 12 or to functional equivalents thereof. Delimitation of the promoter sequence to particular essential regulatory regions can be carried out for example with the aid of search routine to search for promoter elements. Particular promoter elements are often present in increased numbers in the regions relevant for promoter activity. This analysis can be carried out for example with computer programs such as the PLACE program (“Plant Cis-acting Regulatory DNA Elements”; Higo K et al. (1999) Nucl Acids Res 27(1): 297-300), the BIOBASE database “Transfac” (Biologische Datenbanken GmbH, Braunschweig; Wingender E et al. (2001) Nucleic Acids Res 29(1):281-3) or the PlantCARE database (Lescot M et al. (2002) Nucleic Acids Res 30(1):325-7).

The functionally equivalent fragments of one of the promoters of the invention—for example of the promoters described by SEQ ID NO: 1, 2, 3, 4, 5, 6, 7, 8, 9, 10, 11 or 12—preferably comprise at least 200 base pairs, very particularly preferably at least 500 base pairs, most preferably at least 1000 base pairs of the 3′ end of the respective promoter of the invention—for example the promoters described by SEQ ID NO: 1, 2, 3, 4, 5, 6, 7, 8, 9, 10, 11 or 12—the length being calculated from the transcription start (“ATG” codon) upstream in the 5′ direction. Very particularly preferred functional equivalents are the promoter sequences described by SEQ ID NO: 2, 3, 5, 6, 8 or 9. Further functionally equivalent fragments may be generated for example by deleting any 5′-untranslated regions still present. For this purpose, the start of transcription of the corresponding genes can be determined by methods familiar to the skilled worker (such as, for example, 5′-RACE), and the 5′-untranslated regions can be deleted by PCR-mediated methods or endonuclease digestion.

In transgenic expression cassettes of the invention, at least one of the promoters of the invention (e.g. described by SEQ ID NO: 1, 2, 3, 4, 5, 6, 7, 8, 9, 10, 11 or 12) is functionally linked to at least one nucleic acid sequence to be expressed transgenically.

A functional linkage means, for example, the sequential arrangement of one of the promoters of the invention (e.g. described by SEQ ID NO: 1, 2, 3, 4, 5, 6, 7, 8, 9, 10, 11 or 12) with a nucleic acid sequence to be expressed transgenically and, where appropriate, further genetic control sequences such as, for example, a terminator or a polyadenylation sequence in such a way that the promoter is able to fulfill its function in the transgenic expression of the nucleic acid sequence under suitable conditions, and expression of the nucleic acid sequence (i.e. transcription and, where appropriate, translation) takes place. “Suitable conditions” means in this connection preferably the presence of the expression cassette in a plant cell, preferably a plant cell comprised by a floral tissue of a plant.

Arrangements in which the nucleic acid sequence to be expressed transgenically is positioned downstream of one of the promoters of the invention (e.g. described by SEQ ID NO: 1, 2, 3, 4, 5, 6, 7, 8, 9, 10, 11 or 12), so that the two sequences are covalently connected together, are preferred. In this connection, the distance between the promoter sequence and the nucleic acid sequence to be expressed transgenically is preferably fewer than 200 base pairs, particularly preferably less than 100 base pairs, very particularly preferably less than 50 base pairs.

Generation of a functional linkage and generation of a transgenic expression construct can be achieved by means of conventional recombination and cloning techniques as described for example in Maniatis T, Fritsch E F and Sambrook J (1989) Molecular Cloning: A Laboratory Manual, Cold Spring Harbor Laboratory, Cold Spring Harbor (NY), in Silhavy T J, Berman M L and Enquist L W (1984) Experiments with Gene Fusions, Cold Spring Harbor Laboratory, Cold Spring Harbor (NY) and in Ausubel F M et al. (1987) Current Protocols in Molecular Biology, Greene Publishing Assoc. and Wiley Interscience. However, further sequences which have for example the function of a linker with particular restriction enzyme cleavage sites or of a signal peptide may also be positioned between the two sequences. Insertion of sequences may also lead to expression of fusion proteins. It is possible and preferred for the transgenic expression construct, consisting of a linkage of promoter and nucleic acid sequence to be expressed, to be integrated into a vector and be inserted into a plant genome for example by transformation.

However, an expression cassette also means constructs in which one of the promoters of the invention (e.g. described by SEQ ID NO: 1, 2, 3, 4, 5, 6, 7, 8, 9, 10, 11 or 12) is, without necessarily having been functionally linked beforehand to a nucleic acid sequence to be expressed, introduced into a host genome, for example by targeted homologous recombination or random insertion, there undertakes regulatory control over endogenous nucleic acid sequences then functionally linked thereto, and controls the transgenic expression thereof. Insertion of the promoter—for example by a homologous recombination—in front of a nucleic acid coding for a particular polypeptide results in an expression cassette of the invention which controls the expression of the particular polypeptide selectively in the tissues of the flowers. It is also possible for example for the natural promoter of an endogenous gene to be replaced by one of the promoters of the invention (e.g. described by SEQ ID NO: 1, 2, 3, 4, 5, 6, 7, 8, 9, 10, 11 or 12), and for the expression behavior of the endogenous gene to be modified.

A further possibility is also for the promoter to be inserted in such a way that antisense RNA to the nucleic acid coding for a particular polypeptide is expressed. In this way, expression of the particular polypeptide in the organs of the flower is selectively downregulated or switched off.

It is also possible analogously for a nucleic acid sequence which is to be expressed transgenically to be placed—for example by homologous recombination—downstream of the sequence which codes for one of the promoters of the invention (e.g. described by SEQ ID NO: 1, 2, 3, 4, 5, 6, 7, 8, 9, 10, 11 or 12), and which is located in its natural chromosomal context, so as to result in an expression cassette of the invention which controls the expression of the nucleic acid sequence to be expressed transgenically in the floral tissues.

The transgenic expression cassettes of the invention may comprise further genetic control sequences. The term genetic control sequences is to be understood broadly and means all sequences having an influence on the coming into existence or the function of a transgenic expression cassette of the invention. Genetic control sequences modify for example the transcription and translation in prokaryotic or eukaryotic organisms. The transgenic expression cassettes of the invention preferably comprise as additional genetic control sequence a terminator sequence 3′-downstream from the particular nucleic acid sequence to be expressed transgenically, and where appropriate further customary regulatory elements, in each case functionally linked to the nucleic acid sequence to be expressed transgenically.

Genetic control sequences also include further promoters, promoter elements or minimal promoters able to modify the expression-controlling properties. It is thus possible for example through genetic control sequences for tissue-specific expression to take place additionally in dependence on particular stress factors. Corresponding elements are described for example for water stress, abscisic acid (Lam E and Chua N H, J Biol Chem 1991; 266(26):17131-17135) and heat stress (Schoffl F et al. (1989) Mol Gen Genetics 217(2-3):246-53).

A further possibility is for further promoters which make transgenic expression possible in further plant tissues or in other organisms such as, for example, E. coli bacteria to be functionally linked to the nucleic acid sequence to be expressed. Suitable promoters are in principle all plant-specific promoters. Plant-specific promoters means in principle every promoter able to control the expression of genes, in particular foreign genes, in plants or plant parts, plant cells, plant tissues, plant cultures. It is moreover possible for expression to be for example constitutive, inducible or development-dependent. Preference is given to constitutive promoters, tissue-specific promoters, development-dependent promoters, chemically inducible, stress-inducible or pathogen-inducible promoters. Corresponding promoters are generally known to the skilled worker.

Further advantageous control sequences are to be found for example in the promoters of Gram-positive bacteria such as amy and SPO2 or in the yeast or fungal promoters ADC1, MFa, AC, P-60, CYC1, GAPDH, TEF, rp28, ADH.

It is possible in principle for all natural promoters with their regulatory sequences like those mentioned above to be used for the method of the invention. It is additionally also possible for synthetic promoters to be used advantageously.

Genetic control sequences further include also the 5′-untranslated regions, introns or noncoding 3′ region of genes such as, for example, the actin-1 intron, or the Adh1-S introns 1, 2 and 6 (generally: The Maize Handbook, Chapter 116, Freeling and Walbot, Eds., Springer, New York (1994)), preferably the genes with the gene locus At5g33370, At5g22430, At1g26630, At4g35100, At3g04290 and At5g46110 from Arabidopsis thaliana. It is possible to show that such regions may have a significant function in regulating gene expression. Thus, it has been shown that 5′-untranslated sequences are able to enhance the transient expression of heterologous genes. Examples of translation enhancers which may be mentioned are the 5′ leader sequence from tobacco mosaic virus (Gallie et al. (1987) Nucl Acids Res 15:8693-8711) and the like. They may in addition promote tissue specificity (Rouster J et al. (1998) Plant J 15:435-440). The nucleic acid sequences indicated under SEQ ID NO: 1, 4, 7, 10, 11 and 12 in each case represent the promoter region and the 5′-untranslated regions up to the ATG start codon of the respective genes with the gene locus At5g33370, At5g22430, At1g26630, At4g35100, At3g04290 and At5g46110.

The transgenic expression construct may advantageously comprise one or more so-called enhancer sequences functionally linked to the promoter, which make increased transgenic expression of the nucleic acid sequence possible. Additional advantageous sequences can also be inserted at the 3′ end of the nucleic acid sequences to be expressed transgenically, such as further regulatory elements or terminators. The nucleic acid sequences to be expressed transgenically may be present in one or more copies in the gene construct.

Polyadenylation signals suitable as control sequences are plant polyadenylation signals, preferably those which are essentially T-DNA polyadenylation signals from Agrobakterium tumefaciens. Examples of particularly suitable terminator sequences are the OCS (octopine synthase) terminator and the NOS (nopaline synthase) terminator.

Control sequences additionally mean those which make homologous recombination or insertion into the genome of a host organism possible or allow deletion from the genome. In homologous recombination for example the coding sequence of a particular endogenous gene can be specifically replaced by a sequence coding for a dsRNA. Methods such as cre/lox technology permit tissue-specific, and in some circumstances inducible, deletion of the transgenic expression construct from the genome of the host organism (Sauer B (1998) Methods 14(4):381-92). In this case, particular flanking sequences are attached to the target gene (lox sequences), which make later deletion by means of cre recombinase possible.

A transgenic expression cassette and/or the transgenic expression vectors derived therefrom may comprise further functional elements. The term functional element is to be understood broadly and means all elements which have an influence on the generation, replication or function of the transgenic expression constructs of the invention, of the transgenic expression vectors or of the transgenic organisms. Non-limiting examples which may be mentioned are:
a) Selection markers which confer resistance to biocides such as metabolism inhibitors (e.g. 2-deoxyglucose 6-phosphate; WO 98/45456), antibiotics (e.g. kanamycin, G 418, bleomycin, hygromycin) or—preferably—herbicides (e.g. phosphinothricin). Examples of selection markers which may be mentioned are: phosphinothricin acetyltransferases (bar and pat gene), which inactivate glutamine synthase inhibitors, 5-enolpyruvylshikimate-3-phosphate synthases (EPSP synthase genes) which confer resistance to glyphosatr (N-(phosphonomethyl)glycine), glyphosatr-degrading enzymes (gox gene product; glyphosate oxidoreductase), dehalogenases which for example inactivate dalapon (deh gene product), sulfonylurea- and imidazolinone-inactivating acetolactate synthases, and nitrilases which for example degrade bromoxynil (bxn gene product), the aasa gene product which confers resistance to the antibiotic apectinomycin, streptomycin phosphotransferases (SPT) which ensure resistance to streptomycin, neomycin phosphotransferases (NPTII) which confer resistance to kanamycin or geneticidin, the hygromycin phosphotransferases (HPT) which mediate resistance to hygromycin, the acetolactate synthases (ALS) which confer resistance to sulfonylurea herbicides (e.g. mutated ALS variants with, for example, the S4 and/or Hra mutation).
b) Reporter genes which code for easily quantifiable proteins and ensure via an intrinsic color or enzymic activity an assessment of the transformation efficiency or of the location or timing of expression. Very particular preference is given in this connection to reporter proteins (Schenborn E, Groskreutz D. Mol. Biotechnol. 1999; 13(1):29-44) such as the green fluorescence protein (GFP) (Sheen et al. (1995) Plant Journal 8(5):777-784), the chloramphenicol transferase, a luciferase (Ow et al. (1986) Science 234:856-859), the aequorin gene (Prasher et al. (1985) Biochem Biophys Res Commun 126(3):1259-1268), b-galactosidase, with very particular preference for β-glucuronidase (Jefferson et al. (1987) EMBO J 6:3901-3907).
c) Origins of replication which ensure replication of the transgenic expression constructs or transgenic expression vectors of the invention in, for example, E. coli. Examples which may be mentioned are ORI (origin of DNA replication), the pBR322 ori or the P15A ori (Sambrook et al.: Molecular Cloning. A Laboratory Manual, 2^{nd} ed. Cold Spring Harbor Laboratory Press, Cold Spring Harbor, N.Y., 1989).
d) Elements which are necessary for agrobacterium-mediated plant transformation, such as, for example, the right or left border of the T-DNA or the vir region.

“Introduction” includes for the purposes of the invention all methods suitable for introducing a nucleic acid sequence (for example an expression cassette of the invention) directly or indirectly into an organism (e.g. a plant) or a cell, compartment, tissue, organ or propagation material (e.g. seeds or fruits) thereof, or for generating such therein. Direct and indirect methods are included. The introduction can lead to a temporary (transient) presence of said nucleic acid sequence or else to a permanent (stable) presence. Introduction includes for example methods such as transfection, transduction or transformation. The organisms used in the methods are grown or cultured, depending on the host organism, in the manner known to the skilled worker.

Introduction of a transgenic expression cassette of the invention into an organism or cells, tissues, organs, parts or seeds thereof (preferably into plants or plant cells, tissues, organs, parts or seeds) can advantageously be achieved by use of vectors comprising the transgenic expression cassettes. Vectors may be for example plasmids, cosmids, phages, viruses or else agrobacteria. The transgenic expression cassettes can be inserted into the vector (preferably a plasmid vector) via a suitable restriction cleavage site. The resulting vector can be firstly introduced and amplified in E. coli. Correctly transformed E. coli are selected and cultured, and the recombinant vector is isolated by methods familiar to the skilled worker. Restriction analysis and sequencing can be used to check the cloning step. Preferred vectors are those making stable integration of the expression cassette into the host genome possible.

Production of a transformed organism (or of a transformed cell or tissue) requires introduction of the appropriate DNA (e.g. the expression vector) or RNA into the appropriate host cell. A large number of methods is available for this process, which is referred to as transformation (or transduction or transfection) (Keown et al. (1990) Methods in Enzymology 185:527-537). Thus, the DNA or RNA can for example be introduced directly by microinjection or by bombardment with DNA-coated microparticles. The cell can also be permeabilized chemically, for example with polyethylene glycol, so that the DNA is able to enter the cell by diffusion. The DNA can also take place by protoplast fusion with other DNA-containing units such as minicells, cells, lysosomes or liposomes. Electroporation is another suitable method for introducing DNA, in which the cells are reversibly permeabilized by an electrical impulse. Corresponding methods are described (for example in Bilang et al. (1991) Gene 100:247-250; Scheid et al. (1991) Mol Gen Genet 228:104-112; Guerche et al. (1987) Plant Science 52:111-116; Neuhause et al. (1987) Theor Appl Genet 75:30-36; Klein et al. (1987) Nature 327:70-73; Howell et al. (1980) Science 208:1265; Horsch et al. (1985) Science 227:1229-1231; DeBlock et al. (1989) Plant Physiology 91:694-701; Methods for Plant Molecular Biology (Weissbach and Weissbach, eds.) Academic Press Inc. (1988); and Methods in Plant Molecular Biology (Schuler and Zielinski, eds.) Academic Press Inc. (1989)).

Vectors preferred for expression in E. coli are pQE70, pQE60 and pQE-9 (QIAGEN, Inc.); pBluescript vectors, Phagescript vectors, pNH8A, pNH16a, pNH18A, pNH46A (Stratagene Cloning Systems, Inc.); ptrc99a, pKK223-3, pKK233-3, pDR540, pRIT5 (Pharmacia Biotech, Inc.).

Preferred vectors for expression in mammalian cells comprise pWLNE0, pSV2CAT, pOG44, pXT1 and pSG (Stratagene Inc.); pSVK3, pBPV, pMSG and pSVL (Pharmacia Biotech, Inc.). Inducible vectors which may be mentioned are pTet-tTak, pTet-Splice, pcDNA4/TO, pcDNA4/TO /LacZ, pcDNA6/TR, pcDNA4/TO/Myc-His/LacZ, pcDNA4/TO/Myc-His A, pcDNA4/TO/Myc-His B, pcDNA4/TO/Myc-His C, pVgRXR (Invitrogen, Inc.) or the pMAM series (Clontech, Inc.; GenBank Accession No: U02443). These themselves provide the inducible regulatory control element for example for a chemically inducible expression.

Vectors for expression in yeast comprise for example pYES2, pYD1, pTEFI/Zeo, pYES2/GS, pPICZ, pGAPZ, pGAPZalph, pPIC9, pPIC3.5, PHIL-D2, PHIL-SI, pPIC3SK, pPIC9K, and PA0815 (Invitrogen, Inc.).

Cloning vectors and techniques for genetic manipulation of ciliates and algae are known to the skilled worker (WO 98/01572; Falciatore et al. (1999) Marine Biotechnology 1(3):239-251; Dunahay et al. (1995) J Phycol 31:10004-1012).

The methods to be used in principle for the transformation of animal cells or of yeast cells are similar to those for “direct” transformation of plant cells. Methods such as calcium phosphate or liposome-mediated transformation or else electroporation are preferred in particular.

Various methods and vectors for inserting genes into the genome of plants and for regenerating plants from plant tissues or plant cells are known (Plant Molecular Biology and Biotechnology (CRC Press, Boca Raton, Fla.), Chapter 6/7, pp. 71-119 (1993); White F F (1993) Vectors for Gene Transfer in Higher Plants; in: Transgenic Plants, Vol. 1, Engineering and Utilization, editors: Kung and Wu R, Academic Press, 15-38; Jenes B et al. (1993) Techniques for Gene Transfer, in: Transgenic Plants, Vol. 1, Engineering and Utilization, editors: Kung and R. Wu, Academic Press, pp. 128-143; Potrykus (1991) Annu Rev Plant Physiol Plant Molec Biol 42:205-225; Halford N G, Shewry P R (2000) Br Med Bull 56(1):62-73). Those mentioned above are included, for example. In the case of plants, the described methods for the transformation and regeneration of plants from plant tissues or plant cells are used for transient or stable transformation. Suitable methods are, in particular, protoplast transformation by polyethylene glycol-induced DNA uptake, calcium phosphate-mediated transformation, DEAE-dextran-mediated transformation, liposome-mediated transformation (Freeman et al. (1984) Plant Cell Physiol. 29:1353ff; U.S. Pat. No. 4,536,475), biolistic methods with the gene gun (“particle bombardment” method; U.S. Pat. No. 5,100,792; EP-A 0 444 882; EP-A 0 434 616; Fromm M E et al. (1990) Bio/Technology 8(9):833-9; Gordon-Kamm et al. (1990) Plant Cell 2:603), electroporation, incubation of dry embryos in DNA-containing solution, electroporation (EP-A 290 395, WO 87/06614), microinjection (WO 92/09696, WO 94/00583, EP-A 0 331 083, EP-A 0 175 966) or other methods of direct DNA introduction (DE 4 005 152, WO 90/12096, U.S. Pat. No. 4,684,611). Physical methods of DNA introduction into plant cells are reviewed in Oard (1991) Biotech Adv 9:1-11.

In the case of these “direct” transformation methods, no particular requirements need be met by the plasmid used. Simple plasmids such as those of the pUC series, pBR322, M13mp series, pACYC184 etc. can be used. If complete plants are to be regenerated from the transformed cells, it is necessary for an additional selectable marker gene to be present on the plasmid.

Besides these “direct” transformation techniques, it is also possible to carry out a transformation by bacterial infection using agrobacterium (e.g. EP 0 116 718), viral infection using viral vectors (EP 0 067 553; U.S. Pat. No. 4,407,956; WO 95/34668; WO 93/03161), or using pollen (EP 0 270 356; WO 85/01856; U.S. Pat. No. 4,684,611).

The transformation is preferably effected using agrobacteria which comprise disarmed Ti plasmid vectors, exploiting their natural ability to transfer genes to plants (EP-A 0 270 355; EP-A0 116 718).

Agrobacterium transformation is widely used for the transformation of dicots, but is also increasingly being applied to monocots (Toriyama et al. (1988) Bio/Technology 6: 1072-1074; Zhang et al. (1988) Plant Cell Rep 7:379-384; Zhang et al. (1988) Theor Appl Genet 76:835-840; Shimamoto et al. (1989) Nature 338:274-276; Datta et al. (1990) Bio/Technology 8: 736-740; Christou et al. (1991) Bio/Technology 9:957-962; Peng et al. (1991) International Rice Research Institute, Manila, Philippines 563-574; Cao et al. (1992) Plant Cell Rep 11:585-591; Li et al. (1993) Plant Cell Rep 12:250-255; Rathore et al. (1993) Plant Mol Biol 21:871-884; Fromm et al. (1990) Bio/Technology 8:833-839; Gordon-Kamm et al. (1990) Plant Cell 2:603-618; D'Halluin et al. (1992) Plant Cell 4:1495-1505; Walters et al. (1992) Plant Mol Biol 18:189-200; Koziel et al. (1993) Biotechnology 11:194-200; Vasil I K (1994) Plant Mol Biol 25:925-937; Weeks et al. (1993) Plant Physiol 102:1077-1084; Somers et al. (1992) Bio/Technology 10:1589-1594; WO 92/14828; Hiei et al. (1994) Plant J 6:271-282).

The strains mostly used for agrobacterium transformation, Agrobakterium tumefaciens or Agrobakterium rhizogenes, comprise a plasmid (Ti or Ri plasmid) which is transferred to the plant after agrobacterium infection. Part of this plasmid, called T-DNA (transferred DNA), is integrated into the genome of the plant cell. Alternatively, binary vectors (mini-Ti plasmids) can also be transferred to plants and integrated in the genome thereof by agrobacterium.

The use of Agrobakterium tumefaciens for the transformation of plants using tissue culture explants is described (inter alia Horsch R B et al. (1985) Science 225:1229ff.; Fraley et al. (1983) Proc Natl Acad Sci USA 80: 4803-4807; Bevans et al. (1983) Nature 304:184-187). Many Agrobakterium tumefaciens strains are able to transfer genetic material—for example the expression cassettes of the invention—such as, for example, the strains EHA101-[pEHA101], EHA105-[pEHA105], LBA4404-[pAL4404], C58C1-[pMP90] and C58C1-[pGV2260] (Hood et al. (1993) Transgenic Res 2:208-218; Hoekema et al. (1983) Nature 303:179-181; Koncz and Schell (1986) Gen Genet 204:383-396; Deblaere et al. (1985) Nucl Acids Res 13: 4777-4788).

On use of agrobacteria, the expression cassette must be integrated into specific plasmids either into a shuttle or intermediate vector or into a binary vector. Binary vectors, which are able to replicate both in E. coli and in agrobacterium, are preferably used. They normally comprise a selection marker gene and a linker or polylinker, flanked by the right and left T-DNA border sequence. They can be transformed directly into agrobacterium (Holsters et al. (1978) Mol Gen Genet 163:181-187). The agrobacterium acting as host organism in this case should already comprise a plasmid having the vir region. This is necessary for transfer of the T-DNA into the plant cell. An agrobacterium transformed in this way can be used to transform plant cells. The use of T-DNA for transforming plant cells has been intensively investigated and described (EP-A 0 120 516; Hoekema, In: The Binary Plant Vector System, Offsetdrukkerij Kanters B. V., Alblasserdam, Chapter V; An et al. (1985) EMBO J 4:277-287). Various binary vectors are known, and some of them are commercially available, such as, for example, pBI101.2 or pBIN19 (Clontech Laboratories, Inc. USA; Bevan et al. (1984) Nucl Acids Res 12:8711), pBinAR, pPZP200 or pPTV.

The agrobacteria transformed with such a vector can then be used in a known manner for transforming plants, especially crop plants such as, for example, oilseed rape, by for example bathing wounded leaves or pieces of leaf in a solution of agrobacteria and then cultivating in suitable media. Transformation of plants by agrobacteria is described (White F F (1993) Vectors for Gene Transfer in Higher Plants; in Transgenic Plants, Vol. 1, Engineering and Utilization, edited by SD Kung and R Wu, Academic Press, pp. 15-38; Jenes B et al. (1993) Techniques for Gene Transfer, in: Transgenic Plants, Vol. 1, Engineering and Utilization, edited by S. D. Kung and R. Wu, Academic Press, pp. 128-143; Potrykus (1991) Annu Rev Plant Physiol Plant Molec Biol 42:205-225). Transgenic plants which comprise in an integrated way the expression systems of the invention described above can be regenerated in a known manner from the transformed cells of the wounded leaves or pieces of leaf.

Stably transformed cells (i.e. those which integrally comprise the DNA introduced into the DNA of the host cell) can be selected from untransformed ones if a selectable marker is a constituent of the introduced DNA. Any gene able to confer a resistance to a biocide (e.g. an antibiotic or herbicide, see above) can act as marker, for example (see above). Transformed cells which express such a marker gene are able to survive in the presence of concentrations of a corresponding biocide which kill an untransformed wild type. The selection marker permits the selection of transformed cells from untransformed ones (McCormick et al. (1986) Plant Cell Reports 5:81-84). The resulting plants can be grown and hybridized in the usual way. Two or more generations should be cultivated in order to ensure that the genomic integration is stable and heritable.

As soon as a transformed plant cell has been produced, it is possible to obtain a complete plant by using methods known to the skilled worker. These entail, for example, starting from callus cultures, single cells (e.g. protoplasts) or leaf disks (Vasil et al. (1984) Cell Culture and Somatic Cell Genetics of Plants, Vol I, II and III, Laboratory Procedures and Their Applications, Academic Press; Weissbach and Weissbach (1989) Methods for Plant Molecular Biology, Academic Press). The formation of shoot and root from these still undifferentiated callus cell masses can be induced in a known manner. The resulting shoots can be planted out and grown. Corresponding methods are described (Fennell et al. (1992) Plant Cell Rep. 11: 567-570; Stoeger et al (1995) Plant Cell Rep. 14:273-278; Jahne et al. (1994) Theor Appl Genet 89:525-533).

The effectiveness of expression of the transgenically expressed nucleic acids can be estimated for example in vitro by shoot-meristem propagation using one of the selection methods described above. In addition, a change in the type and level of expression of a target gene, and the effect on the phenotype of the plant can be tested on test plants in glasshouse tests.

A further aspect of the invention relates to transgenic organisms transformed with at least one expression cassette of the invention or one vector of the invention, and cells, cell cultures, tissues, parts—such as, for example, in the case of plant organisms leaves, roots etc.—or propagation material derived from such organisms.

By organism, starting or host organisms are meant prokaryotic or eukaryotic organisms such as, for example, microorganisms or plant organisms. Preferred microorganisms are bacteria, yeasts, algae or fungi.

Preferred bacteria are bacteria of the genus Escherichia, Erwinia, Agrobakterium, Flavobacterium, Alcaligenes, Pseudomonas, Bacillus or cyanobacteria, for example of the genus Synechocystis and further bacterial genera described in Brock Biology of Microorganisms Eighth Edition on pages A-8, A-9, A10 and A11.

Microorganisms which are particularly preferred are those able to infect plants and thus transfer the constructs of the invention. Preferred microorganisms are those of the genus Agrobakterium and especially of the species Agrobakterium tumefaciens. Particularly preferred microorganisms are those able to produce toxins (e.g. botulinus toxin), pigments (e.g. carotenoids or flavonoids), antibiotics (e.g. penicillin), phenylpropanoids (e.g. tocopherol), polyunsaturated fatty acids (e.g. arachidonic acid) or vitamins (e.g. vitamin B12).

Preferred yeasts are Candida, Saccharomyces, Hansenula, Phaffia rhodozyma or Pichia.

Preferred fungi are Aspergillus, Trichoderma, Blakeslea, Ashbya, Neurospora, Fusarium, Beauveria or further fungi described in Indian Chem Engr. Section B. Vol 37, No. 1, 2 (1995) on page 15, table 6.

Host or starting organisms preferred as transgenic organisms are in particular plant organisms.

“Plant organism or cells derived therefrom” means in general every cell, tissue, part or propagation material (such as seeds or fruits) of an organism capable of photosynthesis. Included for the purposes of the invention are all genera and species of higher and lower plants of the plant kingdom. Annual, perennial, monocotyledonous and dicotyledonous plants are preferred.

“Plant” means for the purposes of the invention all genera and species of higher and lower plants of the plant kingdom. The term includes the mature plants, seeds, shoots and seedlings, and parts derived therefrom, propagation material (for example tubers, seeds or fruits), plant organs, tissues, protoplasts, callus and other cultures, for example cell or callus cultures, and all other types of groupings of plant cells to functional or structural units. Mature plants means plants at any stage of development beyond seedling. Seedling means a young, immature plant at an early stage of development.

Plant organisms for the purposes of the invention are additionally further photosynthetically active organisms such as, for example, algae, cyanobacteria and mosses. Preferred algae are green algae, such as, for example, algae of the genus Haematococcus, Phaedactylum tricornatum, Pirellula, Volvox or Dunaliella. Synechocystis, Chlamydomonas and Scenedesmus are particularly preferred.

Particularly preferred for the purposes of the method of the invention are plant organisms selected from the group of flowering plants (phylum Anthophyta “angiosperms”). All annual and perennial, monocotyledonous and dicotyledonous plants are included. The plant is preferably selected from the following plant families: Amaranthaceae, Amaryllidaceae, Asteraceae, Berberidaceae, Brassicaceae, Cannabaceae, Caprifoliaceae, Caryophyllaceae, Chenopodiaceae, Compositae, Cruciferae, Cucurbitaceae, Fabaceae, Gentianaceae, Geraniaceae, Illiaceae, Labiatae, Lamiaceae, Leguminosae, Liliaceae, Linaceae, Papaveraceae, Papilionoideae, Liliaceae, Linaceae, Malvaceae, Oleaceae, Orchidaceae, Poaceae, Primulaceae, Ranunculaceae, Rosaceae, Rubiaceae, Saxifragaceae, Scrophulariaceae, Solanaceae, Sterculiaceae, Tetragoniacea, Theaceae, Tropaeolaceae, Umbelliferae and Vitaceae.

The invention is very particularly preferably applied to dicotyledonous plant organisms. Preferred dicotyledonous plants are in particular selected from the dicotyledonous crop plants such as, for example, the following

### 1) Category: Dicotyledonae (Dicots). Preferred Families:

Aceraceae (maples)
Cactaceae (cacti)
Rosaceae (roses, apples, almonds, strawberries)
Salicaceae (willows)
Asteraceae (compositae) especially the genus Lactuca, very especially the species sativa (lettuce), and sunflower, dandelion, Tagetes or Calendula and many others,
Cruciferae (Brassicaceae), especially the genus Brassica, very especially the species napus (oilseed rape), campestris (beet), oleracea (e.g. cabbage, cauliflower or broccoli and other brassica species); and of the genus Arabidopsis, very especially the species thaliana, and cress, radish, canola and many others,
Cucurbitaceae such as melon, pumpkin, cucumber or zucchini and many others,
Leguminosae (Fabaceae) especially the genus Glycine, very especially the species max (soybean), soya and alfalfa, pea, beans, lupin or peanut and many others,
Malvaceae, especially mallow, cotton, edible marshmallow, hibiscus and many others,
Rubiaceae, preferably of the subclass Lamiidae such as, for example, Coffea arabica or Coffea liberica (coffee bush) and many others,
Solanaceae, especially the genus Lycopersicon, very especially the species esculentum (tomato) and the genus Solanum, very especially the species tuberosum (potato) and melongena (eggplant) and the genus Capsicum, very especially the species annum (paprika), and tobacco, petunia and many others,
Sterculiaceae, preferably of the subclass Dilleniidae such as, for example, Theobroma cacao (cocoa bush) and many others,
Theaceae, preferably of the subclass Dilleniidae such as, for example, Camellia sinensis or Thea sinensis (tea bush) and many others,
Umbelliferae (Apiaceae), especially the genus Daucus (very especially the species carota (carrot), Apium (very especially the species graveolens dulce (celeriac), and parsley and many others;
   and flax, hemp, spinach, carrot, sugarbeet and the various tree, nut and vine species, especially banana and kiwi fruit.

However, in addition, monocotyledonous plants are also suitable. These are preferably selected from the monocotyledonous crop plants such as, for example the families
Arecaceae (palms)
Bromeliaceae (pineapple, Spanish moss)
Cyperaceae (sedges)
Liliaceae (lilies, tulips, hyacinths, onions, garlic)
Orchidaceae (orchids)
Poaceae (grasses, bamboos, corn, sugarcane, wheat)
Iridaceae (buckwheat, gladioli, crocuses)

Very particular preference is given to Gramineae such as rice, corn, wheat or other cereal species such as barley, millet, rye, triticale or oats, and the sugarcane, and all species of grasses.

Very especially preferred plants are selected from the plant genera Marigold, Tagetes errecta, Tagetes patula, Acacia, Aconitum, Adonis, Arnica, Aquilegia, Aster, Astragalus, Bignonia, Calendula, Caltha, Campanula, Canna, Centaurea, Cheiranthus, Chrysanthemum, Citrus, Crepis, Crocus, Curcurbita, Cytisus, Delonia, Delphinium, Dianthus, Dimorphotheca, Doronicum, Eschscholtzia, Forsythia, Fremontia, Gazania, Gelsemium, Genista, Gentiana, Geranium, Gerbera, Geum, Grevillea, Helenium, Helianthus, Hepatica, Heracleum, Hisbiscus, Heliopsis, Hypericum, Hypochoeris, Impatiens, Iris, Jacaranda, Kerria, Laburnum, Lathyrus, Leontodon, Lilium, Linum, Lotus, Lycopersicon, Lysimachia, Maratia, Medicago, Mimulus, Narcissus, Oenothera, Osmanthus, Petunia, Photinia, Physalis, Phyteuma, Potentilla, Pyracantha, Ranunculus, Rhododendron, Rosa, Rudbeckia, Senecio, Silene, Silphium, Sinapsis, Sorbus, Spartium, Tecoma, Torenia, Tragopogon, Trollius, Tropaeolum, Tulipa, Tussilago, Ulex, Viola or Zinnia, especially preferably selected from the plant genera Marigold, Tagetes erecta, Tagetes patula, Lycopersicon, Rosa, Calendula, Physalis, Medicago, Helianthus, Chrysanthemum, Aster, Tulipa, Narcissus, Petunia, Geranium, Tropaeolum or Adonis.

Within the framework of the expression cassette of the invention, expression of a particular nucleic acid may, through a promoter having specificity for the floral organs, lead to the formation of sense RNA, antisense RNA or double-stranded RNA in the form of an inverted repeat (dsRNAi). The sense RNA can subsequently be translated into particular polypeptides. It is possible with the antisense RNA and dsRNAi to downregulate the expression of particular genes.

The method of gene regulation by means of double-stranded RNA (“double-stranded RNA interference”; dsRNAi) has been described in animal and plant organisms many times (e.g. Matzke M A et al. (2000) Plant Mol Biol 43:401-415; Fire A et al (1998) Nature 391:806-811; WO 99/32619; WO 99/53050; WO 00/68374; WO 00/44914; WO 00/44895; WO 00/49035; WO 00/63364). Express reference is made to the processes and methods described in the citations indicated.

The specificity of the expression constructs and vectors of the invention for flowers of plants is particularly advantageous. The flower has a function in attracting beneficial insects through incorporation of pigments or synthesis of volatile chemicals.

The natural defense mechanisms of the plant, for example against pathogens, are often inadequate. Introduction of foreign genes from plants, animals or microbial sources may enhance the defenses. Examples are protection against insect damage to tobacco through expression of the Bacillus thuringiensis endotoxin (Vaeck et al. (1987) Nature 328:33-37) or protection of tobacco from fungal attack through expression of a chitinase from beans (Broglie et al. (1991) Science 254:1194-1197).

Cold spells during the flowering period lead to considerable crop losses every year. Targeted expression of protective proteins specifically in the flowering period may provide protection.

For such genetic engineering approaches to be highly efficient it is advantageous for there to be concentrated expression of the appropriate nucleic acid sequence to be expressed transgenically in particular in the outermost layer of the flower. Constitutive expression in the whole plant may make the effect problematic, for example through dilution, or impair the growth of the plant or the quality of the plant product. In addition, there may through constitutive expression be increased switching-off of the transgene (“gene silencing”).

Promoters having specificity for the flower are advantageous in this connection. The skilled worker is aware of a large number of proteins whose recombinant expression in the flower is advantageous. The skilled worker is also aware of a large number of genes through which advantageous effects can likewise be achieved through repression or switching-off thereof by means of expression of a corresponding antisense RNA. Non-restrictive examples of advantageous effects which may be mentioned are: achieving resistance to abiotic stress factors (heat, cold, aridity, increased moisture, environmental toxins, UV radiation) and biotic stress factors (pathogens, viruses, insects and diseases), improving the properties of human and animal foods, improving the growth rate or the yield, achieving a longer or earlier flowing period, altering or enhancing the scent or the coloring of the flowers. Non-restrictive examples of the nucleic acid sequences or polypeptides which can be employed in these applications and which may be mentioned are:
1. Improved UV protection of the flowers of plants through alteration of the pigmentation through expression of particular polypeptides such as enzymes or regulators of flavonoid biosynthesis (e.g. chalcone synthases, phenylalanine ammonium-lyases), of DNA repair (e.g. photolyases; Sakamoto A et al. (1998) DNA Seq 9(5-6):335-40), of isoprenoid biosynthesis (e.g. deoxyxylulose-5-phosphate synthases), of IPP synthesis or of carotenoid biosynthesis (e.g. phytoene synthases, phytoene desaturases, lycopene cyclases, hydroxylases or ketolases). Preference is given to nucleic acids which code for the Arabidopsis thaliana chalcone synthase (GenBank Acc. No.: M20308), the Arabidopsis thaliana 6-4 photolyase (GenBank Acc. No.: BAB00748) or the Arabidopsis thaliana blue-light photoreceptor/photolyase homolog (PHHI) (GenBank Acc. No.: U62549) or functional equivalents thereof.
2. Improved protection of the flower of plants from abiotic stress factors such as aridity, heat or cold, for example through overexpression of the antifreeze polypeptides (e.g. from Myoxocephalus Scorpius; WO 00/00512), of the Arabidopsis thaliana transcription activator CBF1, glutamate dehydrogenases (WO 97/12983, WO 98/11240), of a late embryogenesis gene (LEA), for example from barley (WO 97/13843), calcium-dependent protein kinase genes (WO 98/26045), calcineurins (WO 99/05902), farnesyl transferases (WO 99/06580; Pei Z M et al. (1998) Science 282:287-290), ferritin (Deak M et al. (1999) Nature Biotechnology 17:192-196), oxalate oxidase (WO 99/04013; Dunwell J M (1998) Biotechnology and Genetic Engineering Reviews 15:1-32), DREBIA factor (dehydration response element B 1A; Kasuga M et al. (1999) Nature Biotechnology 17:276-286), genes of mannitol or trehalose synthesis (e.g. trehalose-phosphate synthases; trehalose-phosphate phosphatases, WO 97/42326); or through inhibition of genes such as of trehalase (WO 97/50561). Particular preference is given to nucleic acids which code for the Arabidopsis thaliana transcriptional activator CBFI (Gen-Bank Acc. No.: U77378) or the antifreeze protein from Myoxocephalus octodecemspinosus (GenBank Acc. No.: AF306348) or functional equivalents thereof.
3. Achieving resistance for example to fungi, insects, nematodes and diseases through targeted secretion or accumulation of certain metabolites or proteins in the flower. Examples which may be mentioned are glucosinolates (nematode defense), chitinases or glucanases and other enzymes which destroy the cell wall of parasites, ribosome-inactivating proteins (RIPs) and other proteins of the plant resistance and stress response, like those induced on injury or microbial attack of plants or chemically by, for example, salicylic acid, jasmonic acid or ethylene, lysozymes from non-plant sources such as, for example, T4 lysozyme or lysozme from various mammals, insecticidal proteins such as Bacillus thuringiensis endotoxin, a-amylase inhibitor or protease inhibitors (cowpea trypsin inhibitor), glucanases, lectins (e.g. phytohemagglutinin, snowdrop lectin, wheatgerm agglutinin), RNAses or ribozymes. Particular preference is given to nucleic acids which code for the chit42 endochitinase from Trichoderma harzianum (GenBank Acc. No.: S78423) or for the N-hydroxylating, multifunctional cytochrome P-450 (CYP79) from Sorghum bicolor (GenBank Acc. No.: U32624) or functional equivalents thereof.
4. Achieving defense against or attraction of insects, for example through increased release of volatile scents or messengers through, for example, enzymes of terpene biosynthesis.
5. Achieving an ability to store in floral tissues which normally contain no storage proteins or lipids, with the aim of increasing the yield of these substances, e.g. by expression of an acetyl-CoA carboxylase or of enzymes for esterification of metabolites. Preference is given to nucleic acids which code for the Medicago sativa acetyl-CoA carboxylase (Accase) (GenBank Acc. No.: L25042) or functional equivalents thereof.
6. Expression of transport proteins which improve the uptake of metabolites, nutrients or water into the flower and thus optimize flower growth, metabolite composition or yield, for example through expression of an amino acid transporter which increases the rate of uptake of amino acids, or of a monosaccharide transporter which promotes the uptake of sugars. Preference is given to nucleic acids which code for the Arabidopsis thaliana cationic amino acid transporter (GenBank Acc. No.: X92657) or for the Arabidopsis thaliana monosaccharide transporter (Gen-Bank Acc. No.: AJ002399) or functional equivalents thereof.
7. Expression of genes which bring about an accumulation of fine chemicals, such as of tocopherols, tocotrienols, phenylpropanoids, isoprenoids or carotenoids, in the flower. Examples which may be mentioned are the deoxyxylulose-5-phosphate synthases, phytoene synthases, lycopene b-cyclases and the b-carotene ketolases. Preference is given to nucleic acids which code for the Haematococcus pluvialis NIES-144 (Acc. No. D45881) ketolase or functional equivalents thereof.
8. Modification of wax ester formation or of the composition of the deposited oligosaccharides to improve protection against environmental factors or to improve digestibility on use in feedstuffs or foodstuffs. An example which may be mentioned is overexpression of endo-xyloglucan transferase. Preference is given to nucleic acids which code for the Arabidopsis thaliana endo-xyloglucan transferase (EXGT-AI) (Gen-Bank Acc. No.: AF163819) or functional equivalents thereof.
9. Expression of genes, DNA binding proteins, dsRNA and antisense constructions for altering the flower morphology, the time of flowering and the flower senescence, and the flower metabolism. Preference is given to constructions which increase the number of petals, e.g. through downregulation of AGAMOUS and its homologous genes (Yanofsky M F et al. (1990) Nature 346:35-39), make the time of flowering earlier, e.g. through downregulation of FLOWERING LOCUS C (FLC) (Tadege M et al. (2001) Plant J 28(5):545-53) or later, e.g. through overexpression of FLC and delay senescence, e.g. through conferring a flower-specific ethylene insensitivity.
10. Generation of sterile plants by preventing pollination and/or germination by means of the expression of a suitable inhibitor, for example of a toxin, in flowers.
11. Production of nutraceuticals such as, for example,
   a) carotenoids and/or phenylpropanoids e.g. through optimization of the flowers' own metabolic pathways, e.g. through expression of enzymes and regulators of isoprenoid biosynthesis. Preference is given to nucleic acids which code for the Arabidopsis thaliana chalcone synthase (GenBank Acc. No.: M20308), the Arabidopsis thaliana 6-4 photolyase (GenBank Acc. No.: BAB00748) or the Arabidopsis thaliana blue-light photoreceptor/photolyase homolog (PHHI) (GenBank Acc. No.: U62549) or functional equivalents thereof. Preference is likewise given to nucleic acids which code for enzymes and regulators of isoprenoid biosynthesis such as the deoxyxylulose-5-phosphate synthases and of carotenoid biosynthesis such as the phytoene synthases, lycopene cyclases and ketolases, such as of tocopherols, tocotrienols, phenylpropanoids, isoprenoids or carotenoids, in the flower. Examples which may be mentioned are the deoxyxylulose-5-phosphate synthases, phytoene synthases, lycopene cyclases and the carotene ketolases. Particular preference is given to nucleic acids which code for the Haematococcus pluvialis, NIES-144 (Acc. No. D45881) ketolase or functional equivalents.
   b) polyunsaturated fatty acids such as, for example, arachidonic acid or EPA (eicosapentaenoic acid) or DHA (docosahexaenoic acid) through expression of fatty acid elongases and/or desaturases or production of proteins having improved nutritional value, such as, for example, having a high content of essential amino acids (e.g. the methionine-rich 2S albumin gene of the Brazil nut). Preference is given to nucleic acids which code for the Bertholletia excelsa methionine-rich 2S albumin (GenBank Acc. No.: AB044391), the Physcomitrella patens D6-acyl lipid desaturase (GenBank Acc. No.: AJ222980; Girke et al. (1998) Plant J 15:39-48), the Mortierella alpina D6-desaturase (Sakura-dani et al 1999 Gene 238:445-453), the Caenorhabditis elegans D5-desaturase (Michaelson et al. (1998) FEBS Letters 439:215-218), the Caenorhabditis elegans D5-fatty-acid desaturase (des-5) (GenBank Acc. No.: AF078796), the Mortierella alpina D5-desaturase (Michaelson et al. J Biol Chem 273:19055-19059), the Caenorhabditis elegans D6-elongase (Beaudoin et al. (2000) Proc Natl. Acad. Sci. 97:6421-6426), the Physcomitrella patens A6-elongase (Zank et al. (2000) Biochemical Society Transactions 28:654-657) or functional equivalents thereof.
12. Production of pharmaceuticals such as, for example, antibodies, vaccines, hormones and/or antibiotics as described, for example, in Hood E E & Jilka J M (1999) Curr Opin Biotechnol 10(4):382-6; Ma J K & Vine N D (1999) Curr Top Microbiol Immunol 236:275-92.

Further examples of advantageous genes are mentioned for example in Dunwell J M (2000) Transgenic approaches to crop improvement. J Exp Bot. 51 Spec No: 487-96.

A further aspect of the invention relates to the use of the transgenic organisms of the invention described above, and of the cells, cell cultures, parts—such as, for example, in the case of transgenic plant organisms roots, leaves etc.—and transgenic propagation materials such as seeds or fruits, derived therefrom for producing foodstuffs or feedstuffs, pharmaceuticals or fine chemicals.

Preference is further given to a method for the recombinant production of pharmaceuticals or fine chemicals in host organisms, where a host organism is transformed with one of the expression cassettes described above, and this expression cassette comprises one or more structural genes which code for the desired fine chemical, or catalyze the biosynthesis thereof, the transformed host organism is cultivated, and the desired fine chemical is isolated from the cultivation medium. This method can be applied widely to fine chemicals such as enzymes, vitamins, amino acids, sugars, fatty acids, natural and synthetic flavorings, aromatizing substances and colorants. Production of tocopherols and tocotrienols, and carotenoids such as, for example, astaxanthin is particularly preferred. Cultivation of the transformed host organisms and isolation from the host organisms or from the cultivation medium is accomplished by methods known to the skilled worker. The production of pharmaceuticals such as, for example, antibodies or vaccines is described in Hood E E & Jilka J M (1999) Curr Opin Biotechnol 10 (4)382-6; Ma J K & Vine N D (1999) Curr Top Microbiol Immunol 236:275-92.

### Sequences:

1. SEQ ID NO: 1 2554 bp fragment of promoter and 5′-untranslated region of the Arabidopsis thaliana gene locus At5g33370
2. SEQ ID NO: 2 functionally equivalent fragment (1541 bp) of promoter and 5′-untranslated region of the Arabidopsis thaliana gene locus At5g33370
3. SEQ ID NO: 3 functionally equivalent fragment (668 bp) of promoter and 5′-untranslated region of the Arabidopsis thaliana gene locus At5g33370
4. SEQ ID NO: 4 2103 bp fragment of promoter and 5′-untranslated region of the Arabidopsis thaliana gene locus At5g22430
5. SEQ ID NO: 5 functionally equivalent fragment (1376 bp) of promoter and 5′-untranslated region of the Arabidopsis thaliana gene locus At5g22430
6. SEQ ID NO: 6 functionally equivalent fragment (746 bp) of promoter and 5′-untranslated region of the Arabidopsis thaliana gene locus At5g22430
7. SEQ ID NO: 7 2945 bp fragment of promoter and 5′-untranslated region of the Arabidopsis thaliana gene locus At1g26630
8. SEQ ID NO: 8 functionally equivalent fragment (1628 bp) of promoter and 5′-untranslated region of the Arabidopsis thaliana gene locus At1g26630
9. SEQ ID NO: 9 functionally equivalent fragment (587 bp) of promoter and 5′-untranslated region of the Arabidopsis thaliana gene locus At1 g26630
10. SEQ ID NO: 10 2572 bp fragment of promoter and 5′-untranslated region of the Arabidopsis thaliana gene locus At4g35100
11. SEQ ID NO: 11 2421 bp fragment of promoter and 5′-untranslated region of the Arabidopsis thaliana gene locus At3g04290
12. SEQ ID NO: 12 2345 bp fragment of promoter and 5′-untranslated region of the Arabidopsis thaliana gene locus At5g46110
13. SEQ ID NO: 13 oligonucleotide primer M1as
14. SEQ ID NO: 14 oligonucleotide primer M1s
15. SEQ ID NO: 15 oligonucleotide primer M1ss
16. SEQ ID NO: 16 oligonucleotide primer M1svl
17. SEQ ID NO: 17 oligonucleotide primer M2as
18. SEQ ID NO: 18 oligonucleotide primer M2s
19. SEQ ID NO: 19 oligonucleotide primer M2ss
20. SEQ ID NO: 20 oligonucleotide primer M2svl
21. SEQ ID NO: 21 oligonucleotide primer M3as
22. SEQ ID NO: 22 oligonucleotide primer M3s
23. SEQ ID NO: 23 oligonucleotide primer M3ss
24. SEQ ID NO: 24 oligonucleotide primer M3svl
25. SEQ ID NO: 25 oligonucleotide primer M4as
26. SEQ ID NO: 26 oligonucleotide primer M4s
27. SEQ ID NO: 27 oligonucleotide primer M5as
28. SEQ ID NO: 28 oligonucleotide primer M5s
29. SEQ ID NO: 29 oligonucleotide primer M6as
30. SEQ ID NO: 30 oligonucleotide primer M6s

### EXAMPLES

### General Methods

Oligonucleotides can be chemically synthesized for example in a known manner by the phosphoamidite method (Voet & Voet (1995), 2^{nd} edition, Wiley Press New York, pages 896-897). The cloning steps carried out for the purposes of the present invention, such as, for example, restriction cleavages, agarose gel electrophoresis, purification of DNA fragments, transfer of nucleic acids to nitrocellulose and nylon membranes, linkage of DNA fragments, transformation of E. coli cells, culturing of bacteria, replication of phages and sequence analysis of recombinant DNA, are carried out as described in Sambrook et al. (1989) Cold Spring Harbor Laboratory Press; ISBN 0-87969-309-6. Recombinant DNA molecules are sequenced by the method of Sanger (Sanger et al. (1977) Pro Natl Acad Sci USA 74:5463-5467) using an ABI laser fluorescence DNA sequencer.

In a desiccator, to generate transgenic Arabidopsis plants, Agrobakterium tumefaciens (strain C58C1 pMP90) is transformed with various promoter/GUS vector constructs. The agrobacterial strains are subsequently used for generating transgenic plants. To this end, an individual transformed Agrobacterium colony is incubated overnight at 28° C. in a 4 ml culture (medium: YEB medium with 50 μg/ml Kanamycin and 25 μg/ml Rifampicin). A 400 ml culture in the same medium is subsequently inoculated with this culture, incubated overnight (28° C., 220 rpm) and centrifuged (GSA rotor, 8000 rpm, 20 min). The pellet is resuspended in infiltration medium (½ MS medium; 0.5 g/l MES, pH 5.8; 50 g/l sucrose). The suspension is introduced into a plant box (Duchefa), and 100 ml of SILVET L-77 (with polyalkylene oxide-modified heptamethyltrisiloxane; Osi Specialties Inc., Cat. P030196) was added to a final concentration of 0.02%. The plant box with 8 to 12 plants is exposed to a vacuum for 10 to 15 minutes in a desiccator, followed by spontaneous aeration. This is repeated 2 to 3 times. Thereafter, all plants are planted in plant pots containing moist compost and grown under long-day conditions (16 hours illumination; day-time temperature 22 to 24° C., night-time temperature 19° C.; 65% relative atmospheric humidity). The seeds were harvested after 6 weeks.

### Example 1

### Growth Conditions of the Plants for Tissue-Specific RT-PCR Analysis

To obtain 4- or 7-day old seedlings, in each case approximately 400 seeds (Arabidopsis thaliana ecotype Columbia) are surface-sterilized for 2 minutes with an 80% strength ethanol solution, treated for 5 minutes with a sodium hypochlorite solution (0.5% v/v), washed three times with distilled water and incubated for 4 days at 4° C. in order to ensure uniform germination. Thereafter, the seeds are incubated and Petri dishes containing MS medium (Sigma M5519) with addition of 1% sucrose, 0.5 g/l MES (Sigma M8652), 0.8% Difco-BactoAgar (Difco 0140-01), pH 5.7. The seedlings are grown in a 16-hour light/8-hour dark photoperiod (Philips 58 W/33 white light lamps) at 22° C. and harvested after 4 days after the germination phase had begun.

To obtain roots, 100 seeds are sterilized as described above, incubated for 4 days at 4° C. and then grown in 250 ml flasks containing MS medium (Sigma M5519) with addition of a further 3% sucrose and 0.5 g/l MES (Sigma M8652), pH 5.7. The seedlings are grown in a 16-hour light/8-hour dark photoperiod (Philips 58 W/33 white light lamps) at 22° C., 120 rpm, and harvested after 3 weeks. For all the other plant organs used, the seeds are sown on standard compost (type VM, Manna-Italia, Via S. Giacomo 42, 39050 San Giacomo/Laives, Bolzano, Italy), incubated for 4 days at 4° C. in order to ensure uniform germination, and then grown in a 16-hour light/8-hour dark photoperiod (OSRAM Lumi-lux Daylight 36 W/12 fluorescent tubes) at 22° C. Young rosette leaves are harvested in the 8-leaf stage (after 3 weeks), mature rosette leaves are harvested after 8 weeks shortly before stem development. Inflorescences (Apices) of the elongating stems are harvested shortly after elongation. Stems, stem leaves and flower buds are harvested at developmental stage 12 (Bowmann J (ed.), Arabidopsis, Atlas of Morphology, Springer New York, 1995) before the stamina develop. Opened flowers are harvested at stage 14 immediately after the stamina have developed. Wilting flowers are harvested at stage 15 to 16. The green and yellow pods used had a length of 10 to 13 mm.

### Example 2

### Detection of the Tissue-Specific Expression

To identify the promoter characteristics and the essential elements of the promoter, which account for its tissue specificity, it is necessary to place the promoter and various fragments thereof upstream of what is known as a reporter gene, which makes possible the determination of the expression activity. An example which may be mentioned is the bacterial β-glucuronidase (Jefferson et al. (1987) EMBO J 6:3901-3907). The β-glucuronidase activity can be determined in planta by means of a chromogenic substrate such as 5-bromo-4-chloro-3-indolyl-β-D-glucuronic acid in an activity staining procedure (Jefferson et al. (1987) Plant Mol Biol Rep 5:387-405). To study the tissue specificity, the plant tissue is cut into sections and these are embedded, stained and analyzed as described (for example Bäumlein H et al. (1991) Mol Gen Genet 225:121-128).

The substrate used for the quantitative activity determination of the β-glucuronidase is MUG (methylumbelliferylglucuronide), which is cleaved into MU (methylumbelliferon) and glucuronic acid. Under alkaline conditions, this cleavage can be monitored fluorometrically in a quantitative fashion (excitation at 365 nm, measurement of the emission at 455 nm; SpectroFluorimeter Thermo Life Sciences Fluoroscan) as described (Bustos M M et al. (1989) Plant Gell 1:839-853).

### Example 3

### Cloning of the Promoters

In order to isolate the complete promoters as shown in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 and SEQ ID NO: 12, genomic DNA is extracted from Arabidopsis thaliana (ecotype Landsberg erecta) as described (Galbiati M et al. Funct. Integr. Genomics 2000, 20 1:25-34). The isolated DNA is employed as template DNA in a PCR, using the following oligonucleotide/primer combinations and annealing temperatures:

| | | | | Annealing |
|---|---|---|---|---|
| | Promoter | | | temper- |
| Seq. ID No. | name | Forward primer | Reverse primer | ature |
| Seq. ID No. 1 | M1vl | Seq. ID No. 13 | Seq. ID No. 16 | 54° C. |
| Seq. ID No. 2 | M1l | Seq. ID No. 13 | Seq. ID No. 14 | 47° C. |
| Seq. ID No. 3 | M1s | Seq. ID No. 13 | Seq. ID No. 15 | 54° C. |
| Seq. ID No. 4 | M2vl | Seq. ID No. 17 | Seq. ID No. 20 | 54° C. |
| Seq. ID No. 5 | M2l | Seq. ID No. 17 | Seq. ID No. 18 | 56° C. |
| Seq. ID No. 6 | M2s | Seq. ID No. 17 | Seq. ID No. 19 | 54° C. |
| Seq. ID No. 7 | M3vl | Seq. ID No. 21 | Seq. ID No. 24 | 68° C. |
| Seq. ID No. 8 | M3l | Seq. ID No. 21 | Seq. ID No. 22 | 47° C. |
| Seq. ID No. 9 | M3s | Seq. ID No. 21 | Seq. ID No. 23 | 50° C. |
| Seq. ID No. 10 | M4 | Seq. ID No. 25 | Seq. ID No. 26 | 54° C. |
| Seq. ID No. 11 | M5 | Seq. ID No. 27 | Seq. ID No. 28 | 46° C. |
| Seq. ID No. 12 | M6 | Seq. ID No. 29 | Seq. ID No. 30 | 62° C. |

The amplification is carried out as follows:

80 ng genomic DNA
1× Expand™ Long Template PCR buffer

### 2.5 mM MgCl2,

350 μM each of dATP, dCTP, dGTP and dTTP
300 nM each of each primer
2.5 units Expand™ Long Template Polymerase (Roche Diagnostics).
in a final volume of 25 μl

The following temperature program is used (PTC-100™ Model QfiV; MJ Research, Inc., Watertown, Mass.):
1 cycle of 120 sec at 94° C.
35 cycles of 94° C. for 10 sec, the temperature stated in table 1 for 30 sec and 68° C. for 3 min
1 cycle of 68° C. for 30 min 45

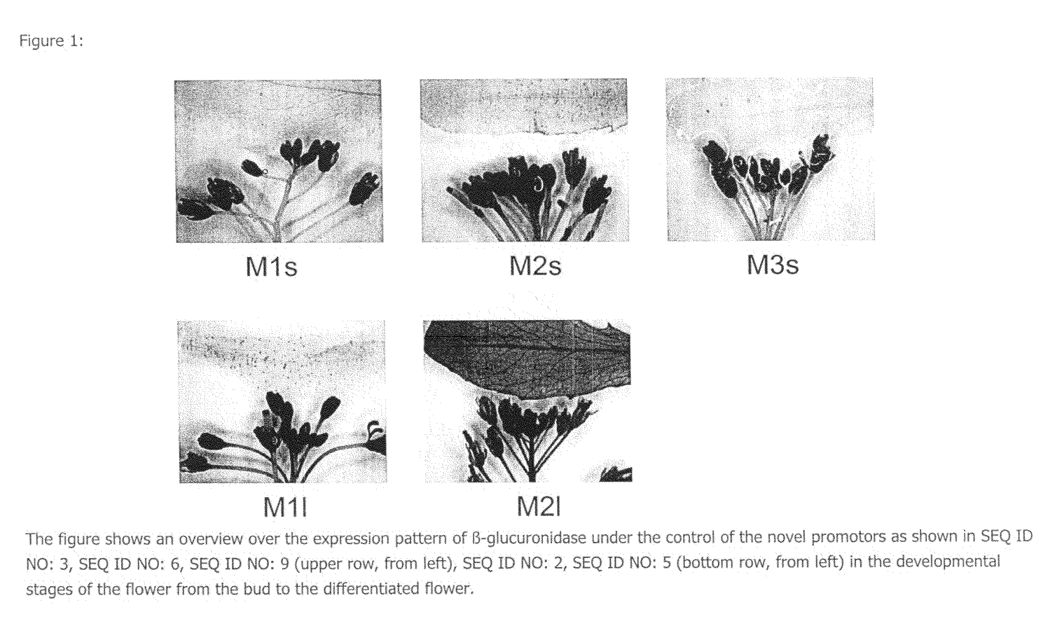

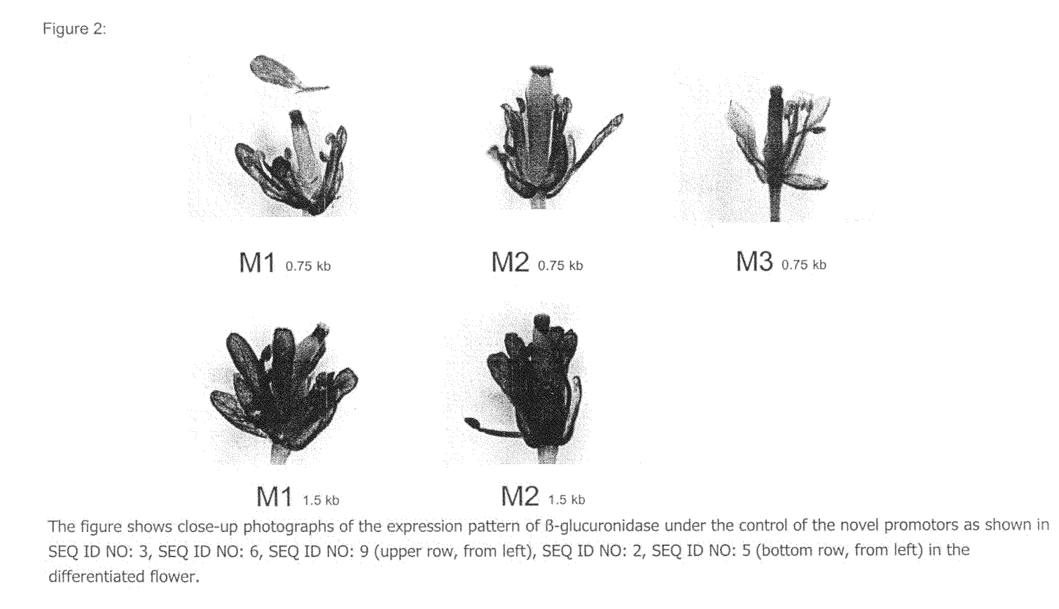

To amplify the fragments, oligonucleotides which bear phosphate residues at their 5′-termini were used as primers. This make possible a direct cloning of the promoters into the vector pS0301 (FIG. 1) which has been opened by the restriction endonuclease SmaI. The vector pS0301 contains the coding sequence of the GUS reporter gene 3 off the SmaI cleavage site. Cloning of the promoter fragments as shown in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 and SEQ ID NO: 12 gave rise to gene fusions of the promoter fragments and of the β-glucuronidase (GUS).

After the Agrobakterium tumefaciens-mediated transformation of these constructs into the genome of Arabidopsis thaliana, the expression of the GUS gene can be visualized by means of histochemical staining methods.

### Example 4

### TAIL PCR

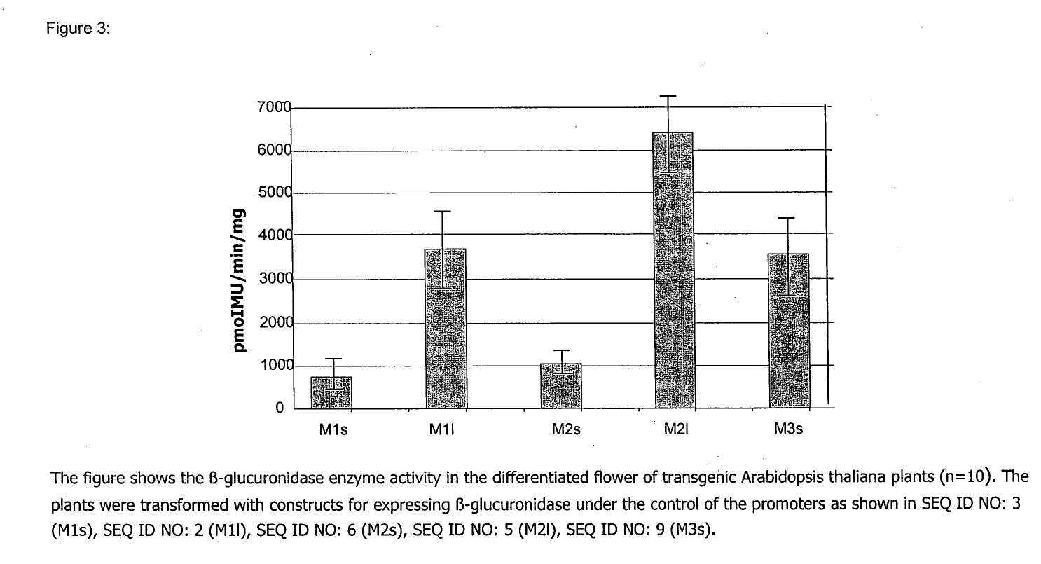

The “TAIL PCR” is performed following an adaptive protocol of the method of Liu et al. (1995) Plant J 8(3):457-463 and Tsugeki et al. (1996) Plant J 10(3):479-489 (cf. FIG. 9). The following master mix (figures per reaction batch) is employed for a first PCR reaction:

11 μl of sterile H2O (double-distilled)
2 μl primer stock solution of the specific primers 1 (5 mM)
3 μl AD2 primer stock solution (20 mM)
2 μl 10×PCR buffer
2 μl 10×dNTP
0.2 μl Taq polymerase

In a PCR vessel, 19 μl of this master mix are pipetted to 1 μl of a genomic DNA preparation of the target organism in question (preparation as described by Galbiati M et al. (2000) Funct Integr Genozides 20(1):25-34)) and mixed thoroughly by pipetting. the primary PCR reaction is carried out under the following conditions:
94° C. for 1 min
four cycles of 94° C. for 10 sec, 62° C. for 1 min and 72° C. for 150 sec
94° C. for 10 sec, 25° C. for 3 min, 0.2° C./sec up to 72° C., and 72° C. for 150 sec
fourteen cycles of 94° C. for 10 sec, 69° C. for 1 min, 72° C. for 150 sec, 94° C. for 10 sec, 68° C. for 1 min, 72° C. for 150 sec, 94° C. for 10 sec, 44° C. for 1 min and 72° C. for 150 sec
72° C. for 5 min, then 4° C. until further use.

The product of the PCR reaction is diluted 1:50, and in each case 1 μl of each dilute sample is employed for a second PCR reaction (secondary PCR). To this end, the following master mix is employed (figures per reaction batch):

12 μl sterile H2O (double-distilled)
2 μl 10×PCR buffer (1.5 mM MgCl2)
2 μl 10×dNTP
2 μl primer stock solution of the specific primers 2 (5 mM)
2 μl AD2 primer stock solution
0.2 μl Taq polymerase

In each case 20.2 μl of the second master mix are added to in each case 1 μl of the 1:50 diluted primary PCR product, and the secondary PCR is carried out under the following conditions:
11 cycles at 94° C. for 10 sec, 64° C. for 1 min, 72° C. for 150 sec, 94° C. for 10 sec, 64° C. for 1 min, 72° C. for 150 sec, 94° C. for 10 sec, 44° C. for 1 min, 72° C. for 150 sec,
72° C. for 5 min, then 4° C. until further use.

The product of the PCR reaction is diluted 1:10, and in each case 1 μl of each dilute sample is employed for a third PCR reaction (tertiary PCR). To this end, the following master mix is employed (figures per reaction batch):

18 μl sterile H2O (double-distilled)
3 μl 10×PCR buffer (1.5 mM MgCl2)
3 μl 10×dNTP
3 μl primer stock solution of the specific primer 3 (5 mM)
3 μl AD2 primer stock solution
0.5 μl Taq polymerase

In each case 30.3 μl of this master mix are added to in each case 1 μl of the 1:10 diluted secondary PCR product, and the tertiary PCR is carried out under the following conditions:
19 cycles at 94° C. for 15 sec, 44° C. for 1 min, 72° C. for 150 sec,
72° C. for 5 min, then 4° C. until further use.

5 μl of each of the products of PCRs 1, 2 and 3 of each sample are separated on a 2% strength agarose gel. Those PCR products which, as a result of the staggered specific primers show the expected size reduction are, if required, isolated from the gel and reamplified with the last-used primer pair and sequenced.

### Reagents:

Taq polymerase 5 U/μl
10×PCR buffer (1.5 mM MgCl2)
10×dNTP stock solution: 2 mM

### Primer:

### Degenerate Random Primers (Stock Solutions 20 μM):

| | |
|---|---|
| AD1: | 5′-NTCGA(G/C)T(A/T)T(G/C)G(A/T)GTT-3′ |
| AD2: | 5′-NGTCGA(G/C)(A/T)GANA(A/T)GAA-3′ |
| AD5: | 5′-(A/T)CAGNTG(A/T)TNGTNCTG-3′ |

### Example 6

### Inverse PCR (iPCR) for the Amplification of Insert-Flanking DNA

The “iPCR” is carried out in accordance with an adapted protocol of the method of Long et al. (1993) PNAS 90:10370:
1. Restriction of approx. 2 μg of genomic DNA with BstYI for approximately 2 hours at 37° C. in a total volume of 50 μl.
2. Ligation of 25 μl of the reaction mixture with 3 U T4-DNA ligase at 15° C. overnight in a total volume of 300 μl.
3. Phenol/chloroform extraction and subsequent chloroform extraction of the ligation mixture. After precipitation with ethanol, take up DNA in 10 μl of sterile H2O (double-distilled).
4. Use 2.5 μl of the DNA solution for the PCR.

### Reaction Mixture:

2.5 μl of the DNA solution
10 μl 10×PCR buffer
2 μl dNTP (in each case 10 mM in the mixture)
5 μl primer 1 (25 pmol)
5 μl primer 2 (25 pmol))
1.5 μl Taq polymerase
74 μl H2O (double-distilled, sterile)
to a total volume of 100 μl
PCR protocol: 4 min for 94° C. Then 35 cycles of 1 min for 94° C., 2 min for 55° C. and 3 min for 72° C. Finally 8 min for 72° C., then 4° C. until further use.

The PCR product is by gel electrophoresis checked, purified and then sequenced as the PCR product.

### Example 5

### Quantification of the Promoter Activity

The substrate used for the quantitative activity determination is R-glucuronidase MUG (methylumbelliferylglucuronide), which is cleaved into MU (methylumbelliferon) and glucuronic acid. Under alkaline conditions, this cleavage can be monitored fluorometrically in quantitative terms (excitation at 365 nm, measurement of the emission at 455 nm; SpectroFluorimeter Thermo Life Sciences Fluoroscan) as described (Bustos M M et al. (1989) Plant Cell 1:839-853).

To measure the GUS enzyme activity, 25 mg of plant tissue were powdered in a mortar and mixed with extraction buffer (50 mM sodium phosphate, pH 7; 10 mM mercaptoethanol; 10 mM EDTA; 0.1% Triton). The insoluble plant material was sedimented by centrifugation (10000 g; 10 min). In each case 10 μl of the supernatant were placed into multititer plates for measuring the GUS enzyme activity. After addition of 90 μl of reaction buffer (extraction buffer+2 mM methylumbelliferyl-β-D-glucuronide), the formation of methylumbelliferon (MU) per minute was determined fluorimetrically (excitation wavelength: 320 nm; emission wavelength: 405 nm) relative to an equilibration series of from 10 to 5000 pmol MU. The data were correlated to the amount of protein which had been determined by the method of Bradford.

1. A method for targeting transgenic expression of nucleic acid sequences in floral tissues of plants, comprising the following steps
I. introducing a transgenic expression cassette into plant cells, wherein the transgenic expression cassette comprises at least the following elements
a) at least one promoter sequence selected from the group of sequences consisting of
i) the promoter sequence as shown in SEQ ID NO: 1, SEQ ID NO: 4, SEQ ID NO: 7, SEQ ID NO: 10, SEQ ID NO: 11 or SEQ ID NO: 12,
ii) functional equivalents of the promoter sequence as shown in SEQ ID NO:2, SEQ ID NO:3, SEQ ID NO: 5, SEQ ID NO:6, SEQ ID NO: 8 or SEQ ID NO: 9 with essentially the same promoter activity as a promoter as shown in SEQ ID NO: 1, SEQ ID NO:4, SEQ ID NO:7, SEQ ID NO: 10, SEQ ID NO: 11 or SEQ ID NO: 12, and
iii) functionally equivalent fragments of the sequences under i) or ii) with essentially the same promoter activity as the promoter sequence as shown in i) or ii)
b) at least one further nucleic acid sequence, and
c) where appropriate further genetic control elements,
wherein at least one promoter sequence and one further nucleic acid sequence are functionally linked with one another, and the further nucleic acid sequence is heterologous in relation to the promoter sequence,
II. selecting transgenic cells which comprise said expression cassette stably integrated into the genome, and
III. regenerating complete plants from said transgenic cells, wherein at least one of the further nucleic acid sequence is expressed in essentially all of the floral tissues.

2. A method of identifying and/or isolating promoters of genes which code for a promoter with specificity for floral tissue, comprising employing at least one nucleic acid sequence or part thereof in the identification and/or isolation procedure, wherein said nucleic acid sequence codes for an amino acid sequence comprising at least part of the sequences of Acc. No. NP 198322, NP 568418, NP 173985, NP 195236, NP 187079, NP 568655 or a variation of these sequences.

3. The method according to claim 2, which is carried out using the polymerase chain reaction and wherein said nucleic acid sequence or part thereof is employed as a primer.

4. A method of preparing a transgenic expression cassette with specificity for floral tissue, comprising the following steps:
I. isolating a promoter with specificity for floral tissue, wherein at least one nucleic acid sequence or a part thereof is employed in the isolation, wherein said nucleic acid sequence codes for an amino acid sequence which comprises at least one sequence of Acc No. NP198322, NP568418, NP173985, NP195236, NP187079, NP568655 or a variation of these sequences; and
II. functionally linking said promoter with a further nucleic acid sequence, wherein said nucleic acid sequence is heterologous in relation to the promoter.

5. The method according to claim 4, which is carried out using the polymerase chain reaction and where said nucleic acid sequence or part thereof is employed as a primer.

6. A transgenic expression cassette for targeting transgenic expression of nucleic acid sequences in floral tissues of plants, comprising
a) at least one promoter sequence selected from the group of sequences consisting of
i) the promoter sequence as shown in SEQ ID NO: 1, SEQ ID NO: 4, SEQ ID NO: 7, SEQ ID NO: 10, SEQ ID NO: 11 or SEQ ID NO: 12,
ii) functional equivalents of the promoter sequence as shown in SEQ ID NO: 2, SEQ ID NO: 3, SEQ ID NO: 5, SEQ ID NO: 6, SEQ ID NO: 8 or SEQ ID NO: 9 with essentially the same promoter activity as a promoter as shown in SEQ ID NO: 1, SEQ ID NO: 4, SEQ ID NO: 7, SEQ ID NO: 10, SEQ ID NO: 11 or SEQ ID NO: 12, and
iii) functionally equivalent fragments of the sequences under i) or ii) with essentially the same promoter activity as the promoter sequence as shown in i) or ii),
b) at least one further nucleic acid sequence, and
c) where appropriate further genetic control elements,
wherein at least one promoter sequence and one further nucleic acid sequence are functionally linked with one another, and the further nucleic acid sequence is heterologous in relation to the promoter sequence.

7. The transgenic expression cassette according to claim 6, wherein
a) the nucleic acid sequence to be expressed is functionally linked with further genetic control sequences, or
b) the expression cassette comprises additional function elements, or
c) a) and b) apply.

8. The transgenic expression cassette according to claim 6, wherein the nucleic acid sequence to be expressed transgenically makes possible
a) the expression of a protein encoded by said nucleic acid sequence, or
b) the expression of a sense-RNA, antisense-RNA or double-stranded RNA encoded by said nucleic acid sequence.

9. The transgenic expression cassette according to claim 6, wherein the nucleic acid sequence to be expressed transgenically is selected from the group of nucleic acid sequences consisting of sequences coding for chalcone synthases, phenylalanine ammoniumlyases, photolyases, deoxyxylulose-5-phosphate synthases, phytoene synthases, phytoene desaturases, lycopene cyclases, hydroxylases, “antifreeze” polypeptides, CBF1 transcription activators, glutamate dehydrogenases, calcium-dependent protein kinases, calcineurin, farnesyl transferases, ferritin, oxalate oxidases, DREB1A factor, trehalose phosphate phosphatases, chitinases, glucanases, ribosome-inactivating protein, lysozymes, Bacillus thuringiensis endotoxins, amylase inhibitors, protease inhibitors, lectins, RNAses, ribozymes, endochitinase, cytochrome P-450, acetyl-CoA carboxylases, amino acid transporters, monosaccharide transporters, lycopene cyclases, carotene ketolases, endoxyloglucan transferases, Δ6-acyl-lipid desaturases, Δ6-desaturases, Δ5-fatty acid desaturase, Δ6-elongases and IPP isomerases.

10. The transgenic expression cassette according to claim 6, wherein the nucleic acid sequence to be expressed transgenically is selected from the group of nucleic acid sequences consisting of sequences described by GenBank Acc.-No.: M20308, BAB00748, U62549, U77378, S78423, U32624, L25042, X92657, AJ002399, D45881, AF163819, AB044391, AJ222980 and AF078796.

11. A transgenic expression vector comprising the expression cassette according to claim 6.

12. A transgenic organism transformed with the expression cassette according to claim 6 or an expression vector comprising said expression cassette.

13. The transgenic organism according to claim 12, selected from the group consisting of bacteria, yeast, fungi, nonhuman, animal and plant organisms, and cells, cell cultures, parts, tissues, organs or propagation material derived from the above.

14. The transgenic organism according to claim 12, selected from the group of the agriculture crop plants.

15. (canceled)

16. A process for producing pharmaceuticals or fine chemicals comprising growing or culturing the transgenic organism of claim 12 or cells, cell cultures, parts, tissues, organs or propagation material derived therefrom and isolating the pharmaceutical or the fine chemical.

17. Feedstuffs, foodstuffs, seeds, pharmaceuticals or fine chemicals produced from the transgenic organism of claim 12, or cell cultures, parts, tissues, organs or propagation material derived therefrom.

18. The fine chemicals of claim 17, wherein the fine chemicals are enzymes, vitamins, amino acids, sugars, saturated or unsaturated fatty acids, natural or synthetic flavorings, aromatizing substances or colorants.
